(19) United States
(12) Patent Application Publication
(10) Pub. No.: US 2018/0344655 A1
(43) Pub. Date: Dec. 6, 2018

(54) FUNCTIONALIZED POLYMERIC PARTICLES FOR TREATMENT OF GLIOMAS

(71) Applicant: Yale University, New Haven, CT (US)

(72) Inventors: William M. Saltzman, New Haven, CT (US); Yang Deng, Edison, NJ (US); Jennifer Saucier-Sawyer, Woburn, MA (US); Alice Gaudin, West Haven, CT (US); Young-Eun Seo, New Haven, CT (US); Eric Song, New Haven, CT (US)

(21) Appl. No.: 15/573,809

(22) PCT Filed: May 11, 2016
(86) PCT No.: PCT/US2016/031890

§ 371 Date: Nov. 13, 2017

Related U.S. Application Data
(60) Provisional application No. 62/232,734, filed on Sep. 25, 2015.
(60) Provisional application No. 62/260,028, filed on Nov. 25, 2015.

(30) Foreign Application Priority Data
May 11, 2015 (US) PCT/US2015/030169
May 11, 2015 (US) PCT/US2015/030187

(51) Int. Cl. A61K 9/51; A61K 9/50; A61K 31/7076; A61K 47/69; A61P 35/00

(52) U.S. Cl. CPC A61K 9/5153; A61K 9/5031; A61P 35/00; A61K 47/6935; A61K 47/6937; A61K 31/7076

(57) ABSTRACT

Nanoparticle compositions including one or more active agents, and strategies for enhanced delivery of the active agents, are provided. In preferred embodiments, the nanoparticles are composed of block copolymers of one or more hydrophobic polymers that form the core, and a hyperbranched polymer that forms a shell or corona. In some embodiments, the particles include an acid-sensitive, poly(amine-co-ester) (PACE) that can increase release of the active agent in acidic environments, for example within endosomes. The compositions can include one or more targeting moieties. Preferred targeting moieties include adenosine agonists and pHLIP which can enhance delivery to tumor cells. Methods of using the compositions to treat diseases and disorders of the central nervous system, for example, brain cancers such as glioma, are also provided.

### CROSS-REFERENCE TO RELATED APPLICATIONS

This application claims the benefit of and priority to pending International Application PCT/US2015/030169 filed May 11, 2015, International Application PCT/US2015/030187 filed May 11, 2015, U.S. Ser. No. 62/232,734 filed Sep. 25, 2015, and U.S. Ser. No. 62/260,028 filed Nov. 25, 2015, the contents of each of which are incorporated by reference in their entireties.

### STATEMENT REGARDING FEDERALLY SPONSORED RESEARCH

This invention was made with government support under Grant 5R01CA149128-04 awarded by the National Institutes of Health. The government has certain rights in the invention.

### FIELD OF THE INVENTION

This application is generally in the field of drug delivery, and more specifically delivery of chemotherapeutics to the brain, especially for the treatment of glioblastoma.

### BACKGROUND OF THE INVENTION

Despite surgical and medical advances, the prognosis for patients with high-grade gliomas, such as glioblastoma multiform (GBM), remains grim (Ostrom, et al. Cancer Treat Res, 163:1-14 (2015)). Although many drug candidates may display interesting in vitro activity, two major obstacles contribute to poor clinical outcomes: (1) drug delivery to the brain is difficult because of fast metabolism and/or rapid clearance, as well as poor permeation through the blood-brain barrier (BBB) (Pardridge, J Cereb Blood Flow Metab, 32: 959-972 (2012)), and (2) 90% of resected tumors recur within 2 cm of the original site due to chemoresistant glioma stem cells (GSCs) that trigger tumor regrowth (Reya, et al., Nature, 414: 105-111 (2001)).

Clinical trials have demonstrated that the BBB can be safely bypassed with direct or regional delivery of therapeutic agents. For example, local implantation of a drug-loaded biodegradable polymer wafer (presently marketed as GLIADEL®), which slowly releases carmustine (BCNU) over a prolonged period, is a safe and effective method for treating GBM. However, use of the GLIADEL® wafer results in only modest improvements in patient survival, typically two months. (H. Brem et al., J Neurosurg 74, 441-446 (1991); H. Brem et al., Lancet 345, 1008-1012 (1995)). These wafers produce high interstitial drug concentrations in the tissue near the implant, but because drugs move from the implant into the tissue by diffusion, penetration into tissue is limited to approximately 1 mm, and does not reach invading GBM stem cells (Fung, et al. Pharm Res 13, 671-682 (1996); Fung et al., Cancer Res 58, 672-684 (1998)).

Convection-enhanced delivery (CED), in which agents are infused into the brain through a catheter under a positive pressure gradient, (Bobo et al., Proc Natl Acad Sci USA 91, 2076-2080 (1994)) has been shown to be clinically safe and feasible (S. Kunwar et al., Neuro Oncol 12, 871-881 (2010); J. H. Sampson et al., Neuro Oncol 10, 320-329 (2008); A. Jacobs et al., Lancet 358, 727-729 (2001)). By creating bulk fluid movement in the brain interstitium, the volume of distribution of the therapeutic agent infused by CED can be much larger than is achievable by diffusion (Morrison et al., American Journal of Physiology 266, R292-R305 (1994)). But CED alone is not sufficient to improve GBM treatment: for example, CED of a targeted toxin in aqueous suspension failed to show survival advantages over GLIADEL® wafers (Kunwar et al., Neuro Oncol 12, 871-881 (2010); Sampson et al., J. neurosurg. 113, 301-309 (2010)). Although CED of drugs in solution results in increased penetration, most drugs have short half-lives in the brain and, as a result, they disappear soon after the infusion stops. (Sampson et al., J. neurosurg. 113, 301-309 (2010); Allard, et al. Biomaterials 30, 2302-2318 (2009)).

Loading of agents into nanocarriers, such as liposomes, micelles, dendrimers, or nanoparticles, can protect them from degradation and clearance. Infusion of nanoparticles into the brain by CED has been previously shown to be feasible, highlighting the necessity of using “brain-penetrating” formulations to penetrate through the brain interstitial spaces (Zhou, et al. Proc Natl Acad Sci USA 110, 11751-11756 (2013); Mastorakos et al., Adv Healthc Mater 4, 1023-1033 (2015), U.S. Published Application No. 2015/0118311). Compared to other carriers, nanoparticles made from the FDA-approved poly(lactide-acid) (PLA) are stable, safe, and tunable to control drug release (Marin et al., Int J Nanomedicine 8, 3071-3090 (2013)). Furthermore, when PLA nanoparticles are coated with hyperbranched polyglycerol (HPG), PLA-HPG nanoparticles (PLA-HPG NPs) significantly resist protein adsorption/cell adhesion (“stealthiness”) and provide versatility and density of attachment of surface ligands (Deng et al., Biomaterals 35, 6595-6602 (2014), Published International Application No. WO 2015/172149). PLA-HPG NPs have the additional advantage that they can be turned into bioadhesive NPs, by converting the vicinal diols of the HPG into aldehydes (—CHO), resulting in PLA-HPG-CHO NPs (Deng et al., Nat Mater 14, 1278-1285 (2015)). However, polymeric NPs in combination with CED produced varying degrees of survival benefits in animal models, and the distribution of the particles beyond the tumor margin may elicit undesirable toxicity and side effects due to the release of the drug in the healthy brain tissue.

Thus it is an object of the invention to provide improved polymer compositions, nanoparticles formed therefrom, and formulations thereof for therapeutic administration into the brain, preferably in combination with convection-enhanced delivery.

It is another object of the invention to provide methods of making improved block co-polymer nanoparticles, loading them with active agents, and using them for treating subjects in need thereof.

It is another object of the invention to provide methods of controlling nanoparticles cellular fate after brain delivery by CED by tuning nanoparticles surface properties, loading them with active agents, and using them for treating subjects in need thereof.

It is a further object of the invention to provide methods of treating brain diseases and disorder, particularly brain cancers such as glioma.

### SUMMARY OF THE INVENTION

Nanoparticle compositions including one or more active agents, and methods and compositions for enhanced delivery of the active agents, are provided. Active agents include, but are not limited to, nucleic acids, particularly inhibitory nucleic acids such as siRNA, and small molecule drugs such as anti-proliferative and pro-apoptotic agents. As discussed in more detail below, the compositions and methods are particularly usefully for delivery of active agents to the central nervous system, particularly the brain, and can be used to treat a variety of diseases and conditions including, but not limited to, brain cancer, disease, injury and disorders. In addition to brain tumors, these compositions and methods are particularly useful for treating neurodegenerative diseases, cerebrovascular diseases, and genetic diseases.

In preferred embodiments, the nanoparticles are composed of polymers or block copolymers of one or more hydrophobic polymers or other hydrophobic molecules, including alkanes, drugs, hydrophobic peptides, PNA, and nucleic acid molecules, that form a core, and a hyperbranched polymer that forms a shell or corona. In a particularly preferred embodiment exemplified in the experiments below, the block copolymer is poly(lactic acid)-hyperbranched polyglycerol (PLA-HPG).

Nanoparticles enter cells primarily through endocytosis, and effective endosomal escape can be important for the biological activity of many intracellular agents. In some embodiments, the particles include an acid-sensitive, polymer core that can increase release of the active agent in acidic environments, for example within endosomes.

The Examples below also show that internalization of stealth particles such as PLA-PEG or PLA-HPG particles is generally lower than that of “sticky” particles such as PLA-HPG-CHO NPs, demonstrating that bioadhesive surface modifications can dramatically enhance the association of NPs with particular cell populations, such as tumor cells. Thus in some embodiments, the particles include an HPG-CHO corona. Coronas of chemistries other than HPG, which have similar densities of aldehyde groups (such as sugar-polymers), can also be used.

The particles can include one or more targeting moieties. For example, in some embodiments, the targeting moiety targets an adenosine receptor. Adenosine receptors, which are expressed on the surface of tumor cells and tumor-associated macrophages, are important regulators of the brain tumor microenvironment, and can make glioma stem cells more sensitive to chemotherapy drugs. Thus in some embodiments, the particles are modified by covalent attachment of an adenosine agonist, such as adenosine, to the surface of nanoparticles to enhance therapeutic efficacy against intracranial tumors.

Another preferred targeting moiety is pHLIP (pH Low Insertion Peptide). pHLIP is a peptide that can selectively translocate cargo across cell membranes at low pH. The tumor-targeting ability of pHLIP is thought to be based on its insertion into membrane in response to environmental acidity, a feature common to solid tumor microenvironments. In some embodiments, the particles include both an adenosine receptor agonist and a pHLIP peptide as targeting moieties. Other targeting ligands include transferrin, EGF, some toxins, rabbi virus peptides, other peptides, antibodies and proteins.

In some embodiments, the particles include a hyperbranched polymer shell in which some of the surface hydroxyl groups are aldehydes and some are functionalized with a targeting moiety. In this way, the particle can be both “sticky” and specifically or selectively targeted to a target cell via a targeting moiety.

Nanoparticles for CED delivery are typically less than about 100 nm in diameter, for example in a range of about 60 to about 90 nm diameter. Additionally or alternatively, additives such as trehalose, other sugars, and other aggregation-reducing materials can be added to any solution including particles, for example, a resuspension solution and/or a pharmaceutical composition for administration to subject in need thereof to enhance CED of the particles.

The addition of ligands at the surface of the nanoparticles that can induce the accumulation of the nanoparticles at the tumor site can increase selective release of the drug only in the tumor environment and reduce off-target toxicity. Despite surgical and medical advances, the prognosis for patients with high-grade gliomas remains grim. To address this challenge, a combination of nanomedicine with convection-enhanced delivery (CED) has been developed. CED of brain-penetrating nanoparticles loaded with chemotherapeutic drugs produces significant increases in the survival of animals with intracranial gliomas. This therapeutic effect is even more striking when the nanoparticles are loaded with drugs that exert high cytotoxic activity against glioma stem cells (GSCs), the most important cells in the development and persistence of brain tumors.

### BRIEF DESCRIPTION OF THE DRAWINGS

FIG. 1 is an illustration of the pH-dependent membrane interaction of pH-low insertion peptide (pHLIP). pHLIP binds to membranes at pH 7.4 unstructured but folds across the bilayer as a transmembrane helix at pH˜6 (Shu, et al., Nature Communications, 6, Article number: 7787 doi:10.1038/ncomms8787 (2015)).

FIG. 2A is a bar graph showing the volume of distribution of PLA nanoparticles and PLA-HPG nanoparticles. FIG. 2B is a bar graph showing the cellular tropism of control (no nanoparticles), PLA nanoparticles, and PLA-HPG nanoparticles illustrated as Normalized Mean Fluorescent Intensity (MFI) in astrocytes, microglia, and neurons.

FIG. 3A is an illustration of PLA-HPG-adenosine (PLA-HPG-Ad) nanoparticles for glioma stem cell sensitization, which can be formed of a combination of PLA-HPG and PLA-HPG-Adenosine conjugated polymers, and loaded with drug (e.g., campthotecine (CPT)) by an emulsion/evaporation process to yield particles. FIG. 3B is a line graph showing % cumulative CPT release over time (hours) from PLA-HPG and PLA-HPG-Ad nanoparticles. FIG. 3C is a schematic illustrating a brain tumor model featuring RG2 cells, and a pre-clinical study design testing convention-enhanced delivery (CED) based delivery of nanoparticle treatment thereof. FIG. 3D is a Kaplan-Meier curve showing % survival of animals treated with phosphate buffered saline (PBS), PLA-HPG nanoparticles, or PLA-HPG-Ad nanoparticles according to the tumor model and assay illustrated in FIG. 4C.

FIG. 4A is an illustration of PLA-HPG nanoparticles for tumor targeting, which can be formed and loaded with dye (e.g., DiD dye) and drug (e.g., paclitaxel (PTX)) by an emulsion/evaporation process to yield particles, and decorated with pHLIP by a Schiff base reaction (PLA-HPG-pHLIP). FIG. 4B is a line graph showing cell viability (%) of a rat glioma cell line (RG2 cells) relative to concentration (μM) of free PTX, PTX-loaded PLA-HPG nanoparticles, and PTX-loaded PLA-HPG-pHLIP nanoparticles in an in vitro cell viability assay. FIG. 4C is a bar graph showing in vitro uptake (Mean Fluorescent Intensity (MFI)) of PLA-HPG nanoparticles and PLA-HPG-pHLIP nanoparticles in RG2 cells at pH 7.4 and pH 6.3. FIG. 4D is a bar graph showing tumor uptake (mean fluorescence intensity (MFI)) of PLA-HPG and PLA-HPG-pHLIP nanoparticles.

FIG. 5 is an illustration of an exemplary multifunctional PLA-HPG nanoparticle, decorated with adenosine and pHILP peptide, and loaded with drug and anti-miR nucleic acids. Increased efficacy can be achieved through controlled release of the particle contents through use of PLA-HPG polymers, sensitization of glioma stem cells by functionalizing the particles with adenosine, and increased internalization in tumor cells by functionalizing the particles with pHILP, particularly when the particles are delivered locally to a brain tumor using convection-enhanced delivery (CED).

FIGS. 6A and 6B are bar graphs showing particle characterization with dynamic light scattering (FIG. 6A) and laser doppler anemometery (FIG. 6B) of hydrodynamic diameters (FIG. 6A) and zeta potential (FIG. 6B) respectively for PLA NPs, PLA-PEG NPs, PLA-HPG NPs, and PLA-HPG-CHO NPs. Size analysis was conducted in water (FIG. 6A) and zeta potential was measured in water and in artificial cerebrospinal fluid (aCSF) (FIG. 6B). Results are presented as mean+SD of N=3 biological replicates. FIG. 6C is a plot showing the particle size (hydrodynamic diameter (nm)) in 37° C. aCSF over 24 h (representative graph of N=3 biological replicates). FIG. 6D is a bar graph showing volumes of distribution (Vd (mm³)) of fluorescently labeled particles infused in healthy Fischer 344 rats via CED (no significance noted using a two sided student's t-test (results are presented as mean±SD of N=3 biological replicates)). FIG. 6E is an illustration showing the distribution of NPs in healthy brain.

FIG. 7 is a bar graph showing mean fluorescence intensity (MFI) of each cell population in the fluorescent particle channel measured by flow cytometry (results are presented as mean±SD of N=5 biological replicates, experiments of same particle type were done on different days to ensure reproducibility of processing, two control brains were harvested each day, statistical analysis was performed using a two sided student's t-test, *p<0.05). The particles were delivered at a concentration of 50 mg/ml and the MFI was normalized by the relative loading of the dye.

FIG. 8A is a series of pie charts showing cell populations determined using flow cytometry in the healthy brain and the tumor bearing brain, after 7 days and 8 days of tumor growth following the implantation of 250,000 RG2-GFP cells (N=6 biological replicates). FIG. 8B is a series of pie charts showing cellular tropism of NPs 4 h and 24 h after CED in the tumor-bearing brain. Absolute amount of fluorescence was derived by multiplying the MFI by the relative number of cells in each population, also measured by flow cytometry. Total area of the pie charts denotes the sum of the absolute fluorescence within the four cell populations, representing the total nanoparticle uptake by these cells, and each slice gives the relative particle uptake for each cell population. Change in uptake between 4 h and 24 h time points show markedly increased selective uptake by tumor cells compared to other cell populations. FIGS. 8C-8F are bar graphs showing cellular tropism of NPs 4 h and 24 h after CED in the tumor-bearing brain. Rats were injected via CED 7 d after implantation of 250,000 RG2-GFP cells. Mean fluorescence intensity (PLA NPs (FIG. 8C), PLA-PEG NPs (FIG. 8D), PLA-HPG NPs (FIG. 8E), PLA-HPG-CHO NPs (FIG. 8F)) of each cell population in the DiA channel was measured with flow cytometry (results are presented as mean±SD of N=5 biological replicates, experiments of same particle type were done on different days to ensure reproducibility of processing, two control brains were harvested each day).

FIG. 9A is a curve fitting of kinetic of association equation to NP uptake in vitro. Similar fits were achieved for all NP/cell combinations with the parameters described (results are presented as mean±SD of N=3 biological replicates, experiment was repeated twice for reproducibility). FIG. 9B is a dot plot showing an association rate, or rate of uptake, which was normalized to the lowest rate (PLA NPs in neurons) (each point is the mean±SD of N=3 biological replicates). FIGS. 9C and 9D are bar graphs showing in vivo MFI values for 4 h and 24 h in healthy (FIG. 9C)) and tumor-bearing (FIG. 9D) brains (in both cases, results are presented as mean±SD of N=5 biological replicates; bars from left to right in each of astrocytes, microglia, and neuron cell types represent: PLA NPs 4 hrs, PLA NPs 24 hrs, PLA-PEG NPs 4 hrs, PLA-PEG NPs 24 hrs, PLA-HPG NPs 4 hrs, PLA-HPG NPs 24 hrs, PLA-HPG-CHO NPs 4 hrs, PLA-HPG-CHO NPs 24 hrs).

### DETAILED DESCRIPTION OF THE INVENTION

### I. Definitions

The term “biocompatible” as used herein refers to one or more materials that are neither themselves toxic to the host (e.g., an animal or human), nor degrade (if the material degrades) at a rate that produces monomeric or oligomeric subunits or other byproducts at toxic concentrations in the host.

The term “biodegradable” as used herein means that the materials degrade or break down into its component subunits, or digestion, e.g., by a biochemical process, of the material into smaller (e.g., non-polymeric) subunits.

The terms “bioactive agent” and “active agent”, as used interchangeably herein, include, without limitation, physiologically or pharmacologically active substances that act locally or systemically in the body. A bioactive agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they localize in a predetermined physiological environment.

As used herein, “controlled release” refers to a release profile of an agent for which the agent release characteristics of time course and/or location are chosen to accomplish therapeutic or convenience objectives not offered by conventional topical formulations.

“Sustained release” as used herein refers to release of a substance over an extended period of time in contrast to a bolus type administration in which the entire amount of the substance is made biologically available at one time.

As used herein, a “multiphasic release profile” refers to an agent release profile having multiple distinct phases or stages, for example, a “biphasic release profile” refers to a release profile having two distinct phases or stages and a “triphasic release profile” refers to a release profile having three distinct phases or stages. Both are examples of multiphasic release.

“Rapid release” as used herein refers to release of an active agent to an environment over a period of seconds to no more than about 60 minutes once release has begun and release can begin within a few seconds or minutes after exposure to an aqueous environment or after completion of a delay period (lag time) after exposure to an aqueous environment.

The term “immediate release” (IR) refers to release of an active agent to an environment over a period of seconds to up to about 30 minutes once release has begun and release begins within a second to no more than about 10 minutes after exposure to an aqueous environment.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

The term “small molecule”, as used herein, generally refers to an organic molecule that is less than about 2000 g/mol in molecular weight, less than about 1500 g/mol, less than about 1000 g/mol, less than about 800 g/mol, or less than about 500 g/mol. Small molecules are non-polymeric and/or non-oligomeric.

The term “copolymer” as used herein, generally refers to a single polymeric material that is comprised of two or more different monomers. The copolymer can be of any form, such as random, block, graft, etc. The copolymers can have any end-group, including capped or acid end groups.

“Hydrophilic,” as used herein, refers to the property of having affinity for water. For example, hydrophilic polymers (or hydrophilic polymer segments) are polymers (or polymer segments) that are primarily soluble in aqueous solutions and/or have a tendency to absorb water. In general, the more hydrophilic a polymer is, the more that polymer tends to dissolve in, mix with, or be wetted by water.

“Hydrophobic,” as used herein, refers to the property of lacking affinity for, or even repelling water. For example, the more hydrophobic a polymer (or polymer segment), the more that polymer (or polymer segment) tends to not dissolve in, not mix with, or not be wetted by water.

Hydrophilicity and hydrophobicity can be spoken of in relative terms, such as, but not limited to, a spectrum of hydrophilicity/hydrophobicity within a group of polymers or polymer segments. In some embodiments wherein two or more polymers are being discussed, the term “hydrophobic polymer” can be defined based on the polymer's relative hydrophobicity when compared to another, more hydrophilic polymer.

The term “lipophilic”, as used herein, refers to compounds having an affinity for lipids.

The term “amphiphilic”, as used herein, refers to a molecule combining hydrophilic and lipophilic (hydrophobic) properties.

The term “microspheres” is art-recognized, and includes substantially spherical colloidal structures formed from biocompatible polymers having a size ranging from about one or greater up to about 1000 microns. In general, “microcapsules,” also an art-recognized term, may be distinguished from microspheres, as formed of a core and shell. The term “microparticles” is also art-recognized, and includes microspheres and microcapsules, as well as structures that may not be readily placed into either of the above two categories, all with dimensions on average of less than about 1000 microns. If the structures are less than about one micron in diameter, then the corresponding art-recognized terms “nanosphere,” “nanocapsule,” and “nanoparticle” may be utilized. In certain embodiments, the nanospheres, nanocapsules and nanoparticles have an average diameter of about 500 nm, 200 nm, 100 nm, 50 nm, 10 nm, or 1 nm.

A composition containing microparticles or nanoparticles may include particles of a range of particle sizes. In certain embodiments, the particle size distribution may be uniform, e.g., within less than about a 20% standard deviation of the mean volume diameter, and in other embodiments, still more uniform, e.g., within about 10% of the median volume diameter.

The term “particle” as used herein refers to any particle formed of, having attached thereon or thereto, or incorporating a therapeutic, diagnostic or prophylactic agent.

“Mean particle size” as used herein, generally refers to the statistical mean particle size (diameter) of the particles in a population of particles. The diameter of an essentially spherical particle may refer to the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering.

“Monodisperse” and “homogeneous size distribution”, are used interchangeably herein and describe a population of nanoparticles or microparticles where all of the particles are the same or nearly the same size. As used herein, a monodisperse distribution refers to particle distributions in which 90% or more of the distribution lies within 15% of the median particle size, more preferably within 10% of the median particle size, most preferably within 5% of the median particle size.

“Branch point”, as used herein, refers to a portion of a polymer-drug conjugate that serves to connect one or more hydrophilic polymer segments to one or more hydrophobic polymer segments.

The term “targeting moiety” as used herein refers to a moiety that localizes to or away from a specific locale. The moiety may be, for example, a protein, nucleic acid, nucleic acid analog, carbohydrate, or small molecule. Said entity may be, for example, a therapeutic compound such as a small molecule, or a diagnostic entity such as a detectable label. Said locale may be a tissue, a particular cell type, or a subcellular compartment. In one embodiment, the targeting moiety directs the localization of an active entity. The active entity may be a small molecule, protein, polymer, or metal. The active entity may be useful for therapeutic, prophylactic, or diagnostic purposes.

The term “reactive coupling group”, as used herein, refers to any chemical functional group capable of reacting with a second functional group to form a covalent bond. The selection of reactive coupling groups is within the ability of the skilled artisan. Examples of reactive coupling groups can include primary amines (—NH₂) and amine-reactive linking groups such as isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters. Most of these conjugate to amines by either acylation or alkylation. Examples of reactive coupling groups can include aldehydes (—COH) and aldehyde reactive linking groups such as hydrazides, alkoxyamines, and primary amines. Examples of reactive coupling groups can include thiol groups (—SH) and sulfhydryl reactive groups such as maleimides, haloacetyls, and pyridyl disulfides. Examples of reactive coupling groups can include photoreactive coupling groups such as aryl azides or diazirines. The coupling reaction may include the use of a catalyst, heat, pH buffers, light, or a combination thereof.

The term “protective group”, as used herein, refers to a functional group that can be added to and/or substituted for another desired functional group to protect the desired functional group from certain reaction conditions and selectively removed and/or replaced to deprotect or expose the desired functional group. Protective groups are known to the skilled artisan. Suitable protective groups may include those described in Greene, T. W. and Wuts, P. G. M., Protective Groups in Organic Synthesis, (1991). Acid sensitive protective groups include dimethoxytrityl (DMT), tert-butylcarbamate (tBoc) and trifluoroacetyl (tFA). Base sensitive protective groups include 9-fluorenylmethoxycarbonyl (Fmoc), isobutyrl (iBu), benzoyl (Bz) and phenoxyacetyl (pac). Other protective groups include acetamidomethyl, acetyl, tert-amyloxycarbonyl, benzyl, benzyloxycarbonyl, 2-(4-biphεnylyl)-2-propyloxycarbonyl, 2-bromobenzyloxycarbonyl, tert-butyl₇ tert-butyloxycarbonyl, 1-carbobenzoxamido-2,2,2-trifluoroethyl, 2,6-dichlorobenzyl, 2-(3,5-dimethoxyphenyl)-2-propyloxycarbonyl, 2,4-dinitrophenyl, dithiasuccinyl, formyl, 4-methoxybenzenesulfonyl, 4-methoxybenzyl, 4-methylbenzyl, o-nitrophenylsulfenyl, 2-phenyl-2-propyloxycarbonyl, α-2,4,5-tetramethylbenzyloxycarbonyl, p-toluenesulfonyl, xanthenyl, benzyl ester, N-hydroxysuccinimide ester, p-nitrobenzyl ester, p-nitrophenyl ester, phenyl ester, p-nitrocarbonate, p-nitrobenzylcarbonate, trimethylsilyl and pentachlorophenyl ester.

“Stealth”, as used herein, refers to the property of nanoparticles. These nanoparticles are not cleared by the mononuclear phagocyte system (MPS) due to the presence of the hydroxyl groups. The stealth particles resist non-specific protein absorption.

“About” is intended to describe values either above or below the stated value in a range of approx. +/−10%. The ranges are intended to be made clear by context, and no further limitation is implied. The use of any and all examples, or exemplary language (e.g., “such as”) provided herein, is intended merely to better illuminate the description and does not pose a limitation on the scope of the description unless otherwise claimed.

The phrase “pharmaceutically acceptable” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “pharmaceutically acceptable salts” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts.

The term “treating” preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain.

The term “therapeutically effective amount” refers to an amount of the therapeutic agent that, when incorporated into and/or onto particles described herein, produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation. In some embodiments, the term “effective amount” refers to an amount of a therapeutic agent or prophylactic agent to reduce or diminish the symptoms of one or more diseases or disorders of the brain, such as reducing tumor size (e.g., tumor volume) or reducing or diminishing one or more symptoms of a neurological disorder, such as memory or learning deficit, tremors or shakes, etc. In still other embodiments, an “effective amount” refers to the amount of a therapeutic agent necessary to repair damaged neurons and/or induce regeneration of neurons.

The terms “incorporated” and “encapsulated” refers to incorporating, formulating, or otherwise including an active agent into and/or onto a composition that allows for release, such as sustained release, of such agent in the desired application. The terms contemplate any manner by which a therapeutic agent or other material is incorporated into a polymer matrix, including for example: attached to a monomer of such polymer (by covalent, ionic, or other binding interaction), physical admixture, enveloping the agent in a coating layer of polymer, and having such monomer be part of the polymerization to give a polymeric formulation, distributed throughout the polymeric matrix, appended to the surface of the polymeric matrix (by covalent or other binding interactions), encapsulated inside the polymeric matrix, etc. The term “co-incorporation” or “co-encapsulation” refers to-the incorporation of a therapeutic agent or other material and at least one other therapeutic agent or other material in a subject composition.

More specifically, the physical form in which any therapeutic agent or other material is encapsulated in polymers may vary with the particular embodiment. For example, a therapeutic agent or other material may be first encapsulated in a microsphere and then combined with the polymer in such a way that at least a portion of the microsphere structure is maintained. Alternatively, a therapeutic agent or other material may be sufficiently immiscible in the polymer that it is dispersed as small droplets, rather than being dissolved, in the polymer.

### II. Core-Shell Particles

The particles can be particles having a core formed of a hydrophobic or poly(amine-co-ester) or poly(amine-co-amide) polymer, and optionally, but preferably a shell formed of a hyperbranched polymer. The core-shell particles can be formed by a co-block polymer.

A. Core

1. Core Polymers

a. Hydrophobic Polymers or Other Molecules

The core of the particles can be formed of or contains one or more hydrophobic materials, typically polymers (e.g., homopolymer, copolymer, terpolymer, etc.) or other hydrophobic molecules such as alkanes, drugs, hydrophobic peptides, PNA, and nucleic acid molcules. The material may be biodegradable or non-biodegradable. In some embodiments, the one or more materials are one or more biodegradable polymers.

In general, synthetic polymers are preferred, although natural polymers may be used and have equivalent or even better properties, especially some of the natural biopolymers which degrade by hydrolysis, such as some of the polyhydroxyalkanoates. Representative synthetic polymers are: poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulfate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers and blends thereof. Examples of preferred natural polymers include proteins such as albumin, collagen, gelatin and prolamines, for example, zein, and polysaccharides such as alginate, cellulose derivatives and polyhydroxyalkanoates, for example, polyhydroxybutyrate. As used herein, “derivatives” include polymers having substitutions, additions of chemical groups and other modifications routinely made by those skilled in the art.

Examples of preferred non-biodegradable polymers include ethylene vinyl acetate, poly(meth)acrylic acid, polyamides, copolymers and mixtures thereof.

In certain embodiments, the hydrophobic polymer is an aliphatic polyester. In preferred embodiments, the hydrophobic polymer is polyhydroxyester such as poly(lactic acid), poly(glycolic acid), or poly(lactic acid-co-glycolic acid). The particles are designed to release molecules to be encapsulated or attached over a period of days to weeks. Factors that affect the duration of release include pH of the surrounding medium (higher rate of release at pH 5 and below due to acid catalyzed hydrolysis of PLGA) and polymer composition. Aliphatic polyesters differ in hydrophobicity and that in turn affects the degradation rate. The hydrophobic poly (lactic acid) (PLA), more hydrophilic poly (glycolic acid) PGA and their copolymers, poly (lactide-co-glycolide) (PLGA) have different release rates. The degradation rate of these polymers, and often the corresponding drug release rate, can vary from days (PGA) to months (PLA) and is easily manipulated by varying the ratio of PLA to PGA. The core can be formed of copolymers including amphiphilic copolymers such as PLGA-PEG or PLURONICS (block copolymers of polyethylene oxide-polypropylene glycol) but this may decrease the benefit of the polyglycerol molecules discussed below.

Other materials may also be incorporated including lipids, fatty acids, and phospholipids. These may be dispersed in or on the particles, or interspersed with the polyglycerol coatings discussed below.

In particular embodiments, the core is formulated of poly-lactic acid (PLA); poly-D-L-glycolide (PLG); poly-D-L-lactide-co-glycolide (PLGA); and poly-cyanoacrylate (PCA); poly-ε-caprolactone (PCL); poly-alkyl-cyano-acrylates (PAC); chitosan (a modified natural carbohydrate polymer prepared by the partial N-deacetylation of the crustacean-derived natural biopolymer chitin); gelatin (a poly-ampholyte consisting of both cationic and anionic groups along with a hydrophilic group); or combinations thereof.

b. Poly(amine-co-ester)s and Poly(amine-co-amides)

The core of the particles can be formed of or contain one or more poly(amine-co-ester), poly(amine-co-amide), or a combination thereof. In some embodiments, the content of a hydrophobic monomer in the polymer is increased relative the content of the same hydrophobic monomer when used to form polyplexes. Increasing the content of a hydrophobic monomer in the polymer forms a polymer that can form solid core nanoparticles in the presence of nucleic acids, including RNA's. Unlike polyplexes, these particles are stable for long periods of time during incubation in buffered water, or serum, or upon administration (e.g., injection) into animals. They also provide for a sustained release of nucleic acids (e.g., siRNA) which leads to long term activity (e.g., siRNA mediate-knockdown).

The polymers can have the general formula:

((A)ₓ-(B)_{y}—(C)_{q}-(D)_{w}-(E)_{f})ₕ,
wherein A, B, C, D, and E independently include monomeric units derived from lactones (such as pentadecalactone), a polyfunctional molecule (such as N-methyldiethanolamine), a diacid or diester (such as diethylsebacate), or polyalkylene oxide (such as polyethylene glycol). In some aspects, the polymers include at least a lactone, a polyfunctional molecule, and a diacid or diester monomeric units. In general, the polyfunctional molecule contains one or more cations, one or more positively ionizable atoms, or combinations thereof. The one or more cations are formed from the protonation of a basic nitrogen atom, or from quaternary nitrogen atoms.

In general, x, y, q, w, and f are independently integers from 0-1000, with the proviso that the sum (x+y+q+w+f) is greater than one. h is an integer from 1 to 1000.

The percent composition of the lactone can be between about 30% and about 100%, calculated as the mole percentage of lactone unit vs. (lactone unit+diester/diacid). Expressed in terms of molar ratio, the lactone unit vs. (lactone unit+diester/diacid) content is between about 0.3 and about 1. Preferably, the number of carbon atoms in the lactone unit is between about 10 and about 24. In some embodiments, the number of carbon atoms in the lactone unit is between about 12 and about 16. In some embodiments, the number of carbon atoms in the lactone unit is 12 (dodecalactone), 15 (pentadecalactone), or 16 (hexadecalactone).

The molecular weight of the lactone unit in the polymer, the lactone unit's content of the polymer, or both, influences the formation of solid core nanoparticles.

Suitable polymers are disclosed in WO 2013/082529 and U.S. Pat. No. 9,272,043. For example, in some embodiments, the polymer has the formula:

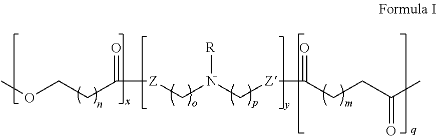

wherein n is an integer from 1-30, m, o, and p are independently an integer from 1-20, x, y, and q are independently integers from 1-1000, Z and Z′ are independently O or NR′, wherein R and R′ are independently hydrogen, substituted or unsubstituted alkyl, or substituted or unsubstituted aryl. Examples of R and R′ groups include, but are not limited to, hydrogen, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl, phenyl, naphthalyl, anthracenyl, phenanthryl, chrysenyl, pyrenyl, tolyl, xylyl, etc. In particular embodiments, the values of x, y, and q are such that the weight average molecular weight of the polymer is greater than 5,000 Daltons. The polymer can be prepared from one or more lactones, one or more amine-diols, triamines, or hydroxy diamines, and one or more diacids or diesters. In those embodiments where two or more different lactone, diacid or diester, and/or triamine, amine-diol, or hydroxy diamine monomers are used, the values of n, o, p, and/or m can be the same or different.

The percent composition of the lactone unit is between about 30% and about 100%, calculated lactone unit vs. (lactone unit+diester/diacid). Expressed in terms of a molar ratio, the lactone unit vs. (lactone unit+diester/diacid) content is between about 0.3 and about 1, i.e., x/(x+q) is between about 0.3 and about 1. Preferably, the number of carbon atoms in the lactone unit is between about 10 and about 24, more preferably the number of carbon atoms in the lactone unit is between about 12 and about 16. Most preferably, the number of carbon atoms in the lactone unit is 12 (dodecalactone), 15 (pentadecalactone), or 16 (hexadecalactone).

In some embodiments, Z and Z′ are O. In some embodiments, Z is O and Z′ is NR′, or Z is NR′ and Z′ is O, wherein R′ is hydrogen, substituted or unsubstituted alkyl, or substituted or unsubstituted aryl. Examples of R′ include, but are not limited to, hydrogen, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl, phenyl, naphthalyl, anthracenyl, phenanthryl, chrysenyl, pyrenyl, tolyl, xylyl, etc.

In some embodiments, Z and Z′ are 0 and n is an integer from 1-24, such 4, 10, 13, or 14.

In some embodiments, Z and Z′ are 0, n is an integer from 1-24, such 4, 10, 13, or 14, and m is an integer from 1-10, such as 4, 5, 6, 7, or 8.

In some embodiments, Z and Z′ are 0, n is an integer from 1-24, such 4, 10, 13, or 14, m is an integer from 1-10, such as 4, 5, 6, 7, or 8, and o and p are the same integer from 1-6, such 2, 3, or 4.

In some embodiments, Z and Z′ are 0, n is an integer from 1-24, such 4, 10, 13, or 14, m is an integer from 1-10, such as 4, 5, 6, 7, or 8, and R is alkyl, such as methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl, or aryl, such as phenyl, naphthalyl, anthracenyl, phenanthryl, chrysenyl, pyrenyl, tolyl, or xylyl.

In certain embodiments, n is 14 (e.g., pentadecalactone, PDL), m is 7 (e.g., diethylsebacate, DES), o and p are 2 (e.g., N-methyldiethanolamine, MDEA). In certain embodiments, n, m, o, and p are as defined above, and PEG is incorporated as a monomer.

In particular embodiments, the values of x, y, and q are such that the weight average molecular weight of the polymer is greater than 5,000 Daltons.

The polymer can be prepared from one or more substituted or unsubstituted lactones, one or more substituted or unsubstituted amine-diols (Z and Z′═O), triamines (Z and Z′═NR′), or hydroxy-diamines (Z═O, and Z′═NR′, or vice versa) and one or more substituted or unsubstituted diacids or diesters. In those embodiments where two or more different lactone, diacid or diester, and/or triamine, amine-diol, or hydroxy diamine monomers are used, than the values of n, o, p, and/or m can be the same or different.

The monomer units can be substituted at one or more positions with one or more substituents. Exemplary substituents include, but are not limited to, alkyl groups, cyclic alkyl groups, alkene groups, cyclic alkene groups, alkynes, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, nitro, heterocyclyl, aralkyl, or an aromatic or heteroaromatic moiety.

The polymer is preferably biocompatible. Readily available lactones of various ring sizes are known to possess low toxicity: for example, polyesters prepared from small lactones, such as poly(caprolactone) and polyp-dioxanone) are commercially available biomaterials which have been used in clinical applications. Large (e.g., C₁₆-C₂₄) lactones and their polyester derivatives are natural products that have been identified in living organisms, such as bees. Lactones containing ring carbon atoms between 16 and 24 are specifically contemplated and disclosed.

In other embodiments, the polymer is biocompatible and biodegradable. The nucleic acid(s) encapsulated by and/or associated with the particles can be released through different mechanisms, including diffusion and degradation of the polymeric matrix. The rate of release can be controlled by varying the monomer composition of the polymer and thus the rate of degradation. For example, if simple hydrolysis is the primary mechanism of degradation, increasing the hydrophobicity of the polymer may slow the rate of degradation and therefore increase the time period of release. In all case, the polymer composition is selected such that an effective amount of nucleic acid(s) is released to achieve the desired purpose/outcome.

The polymers can further include one or more blocks of an alkylene oxide, such as polyethylene oxide, polypropylene oxide, and/or polyethylene oxide-co-polypropylene oxide. The structure of a PEG-containing polymer is shown below:

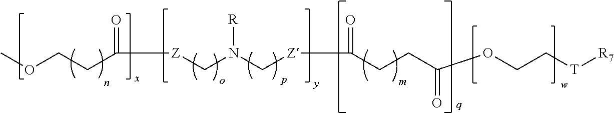

wherein n is an integer from 1-30, m, o, and p are independently an integer from 1-20, x, y, q, and w are independently integers from 1-1000, Z and Z′ are independently O or NR′, wherein R and R′ are independently hydrogen, substituted or unsubstituted alkyl, or substituted or unsubstituted aryl, wherein T is oxygen or is absent, and wherein R₇ is hydrogen, alkyl, substituted alkyl, aryl, substituted alkyl, cycloalkyl, substituted cycloalkyl, maleimide, amine, thiol, N-hydroxysuccinimide ester, azide, acrylate, methacrylate, alkyne, hydroxide, or isocynate. In particular embodiments, the values of x, y, q, and w are such that the weight average molecular weight of the polymer is greater than 5,000 Daltons. Examples of R and R′ groups include, but are not limited to, hydrogen, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl, phenyl, naphthalyl, anthracenyl, phenanthryl, chrysenyl, pyrenyl, tolyl, xylyl, etc.

The structure of a PEG-containing copolymer is shown below:

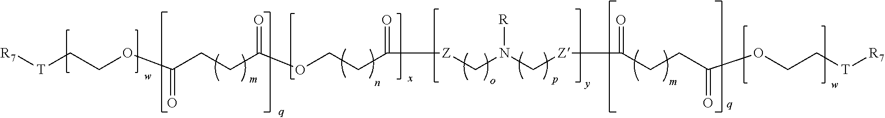

wherein n is an integer from 1-30, m, o, and p are independently an integer from 1-20, x, y, q, and w are independently integers from 1-1000, Z and Z′ are independently O or NR′, wherein R and R′ are independently hydrogen, substituted or unsubstituted alkyl, or substituted or unsubstituted aryl, wherein T is oxygen or is absent, and wherein R₇ is hydrogen, alkyl, substituted alkyl, aryl, substituted alkyl, cycloalkyl, substituted cycloalkyl, maleimide, amine, thiol, N-hydroxysuccinimide ester, azide, acrylate, methacrylate, alkyne, hydroxide, or isocynate. In particular embodiments, the values of x, y, q, and w are such that the weight average molecular weight of the polymer is greater than 5,000 Daltons. Examples of R and R′ groups include, but are not limited to, hydrogen, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl, phenyl, naphthalyl, anthracenyl, phenanthryl, chrysenyl, pyrenyl, tolyl, xylyl, etc.

The blocks of polyalkylene oxide can located at the termini of the polymer (i.e., by reacting PEG having one hydroxy group blocked, for example, with a methoxy group), within the polymer backbone (i.e., neither of the hydroxyl groups are blocked), or combinations thereof.

2. Core Size

The core may vary in size or the core may be formed of two or more layers of hydrophobic material containing the agent, so that the site, duration and manner of release of the active agents are controlled.

3. Core for Controlled Release of Agents

In other embodiments, the core may be formed for extended release of the active agent, so that the active agent is not released, or released within 2, 4, 8, or 24 hours following administration. In other embodiments, the core may be formed of two or more layers of hydrophobic material, each layer containing one or more different agents, and each layer releasing the one or more different agents at specific times to provide for controlled release of the agent.

Delayed release, extended release, and pulsatile release and their combinations are types of controlled release. In preferred formulations, nanoparticle core includes a combination of extended release components, rapid release components, immediate release components, and delayed release components to provide the desired release profile and/or pharmacokinetic parameters. The formulations can have nanoparticles with cores of multiphasic release profile. For example, an agent can be formulated into nanoparticle or microparticle core with an extended release polymer or matrix. The core can be coated with one or more immediate release and/or rapid release dosing layers containing additional agent providing release of the agent at certain times. The rapid release dosing layers can optionally have a delayed release or be coated with a delayed release polymer coating.

a. Extended Release

Cores of particles for extended release of the agent are generally prepared as diffusion or osmotic systems, which are known in the art. A diffusion system typically consists of one of two types of devices, a reservoir or a matrix, and is well known and described in the art. The matrix devices are generally prepared by compressing the agent with a slowly dissolving polymer carrier into the core. The three major types of materials used in the preparation of matrix devices are hydrodphobic polymers, hydrophilic polymers, and fatty compounds. Polymeric matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such ashydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain embodiments, the polymer material is a pharmaceutically acceptable acrylic polymer, including, but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers. In certain embodiments, the acrylic polymer is comprised of one or more ammonio methacrylate copolymers. Animonio methacrylate copolymers are well known in the art, as fully polymerized copolymers of acrylic and methacrylic acid esters with a low content of quaternary ammonium groups.

In one embodiment, the acrylic polymer is an acrylic resin lacquer such as that which is commercially available from Rohm Pharma under the tradename EUDRAGIT®. In other embodiments, the acrylic polymer may be a mixture of two acrylic resin lacquers commercially available from Rohm Pharma under the tradenames EUDRAGIT® RL30D and EUDRAGIT® RS30D, respectively. EUDRAGIT RL30D and EUDRAGIT® RS30D are copolymers of acrylic and methacrylic esters with a low content of quaternary ammonium groups, the molar ratio of ammonium groups to the remaining neutral (meth)acrylic esters being 1:20 in EUDRAGIT® RL30D and 1:40 in EUDRAGIT RS30D. The mean molecular weight is about 150,000. EUDRAGIT® S-100 and EUDRAGIT® L-100 are also preferred. The code designations RL (high permeability) and RS (low permeability) refer to the permeability properties of these agents. EUDRAGIT® RL/RS mixtures are insoluble in water and in digestive fluids. However, multiparticulate systems formed to include the same are swellable and permeable in aqueous solutions and digestive fluids.

The polymers such as EUDRAGIT® RL/RS may be mixed together in any desired ratio in order to ultimately obtain an extended-release core having a desirable dissolution profile. Desirable sustained-release multiparticulate systems may be obtained, for instance, from 100% EUDRAGIT®, 50% EUDRAGIT RL and 50% EUDRAGIT® RS, and 10% EUDRAGIT® RL and 90%: EUDRAGIT® 90% RS. One skilled in the art will recognize that other acrylic polymers may also be used, such as, for example, EUDRAGIT® L.

Alternatively, extended release components can be prepared using osmotic systems or by applying a semi-permeable coating to the core. In the latter case, the desired agent release profile can be achieved by combining low permeable and high permeable coating materials in suitable proportion.

In another embodiment, the agent is dispersed in a matrix material which gels or emulsifies upon contact with an aqueous medium, such as physiological fluids. In the case of gels, the matrix swells entrapping the active agents, which are released slowly over time by diffusion and/or degradation of the matrix material.

b. Delayed Release Components

Delayed release formulations can be created by coating agents and/or cores with a polymer film which is insoluble in the acidic environments and soluble in the neutral environments. Such pH dependent polymers include, but are not limited to, methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), methyl methacrylate-methacrylic acid copolymers, sodium alginate and stearic acid.

B. Shell or Corona

The particles typically include a shell, corona or coating of or containing hyperbranched polymers (HP). Suitable polymers for forming the shell or corona include biodegradable polymeric molecules, such as polyglycerols, polypeptides, oligonucleotides, polysaccharides, and fatty acids. Hyperbranched polyglycerol (HPG) is an exemplary hyperbranched polymer.

1. HPG

In preferred embodiments, the polymer is hyperbranched polyglycerol (HPG), a highly branched polyol containing a polyether scaffold. Hyperbranched polyglycerol can be prepared using techniques known in the art. It can be formed from controlled etherification of glycerol via cationic or anionic ring opening multi-branching polymerization of glycidol. For example, an initiator having multiple reactive sites is reacted with glycidol in the presence of a base to form hyperbranched polyglycerol (HPG). Suitable initiators include, but are not limited to, polyols, e.g., triols, tetraols, pentaols, or greater and polyamines, e.g., triamines, tetraamines, pentaamines, etc. In one embodiment, the initiator is 1,1,1-trihydroxymethyl propane (THP).

A formula for hyperbranched polyglycerol as described in EP 2754684 is

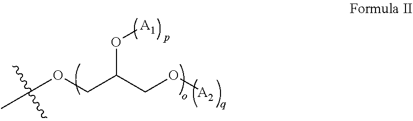

wherein o, p and q are independently integers from 1-100,
wherein A₁ and A₂ are independently

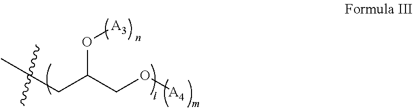

wherein 1, m and n are independently integers from 1-100.
wherein A₃ and A₄ are defined as A₁ and A₂, with the proviso that A₃ and A₄ are hydrogen, n and m are each 1 for terminal residues.

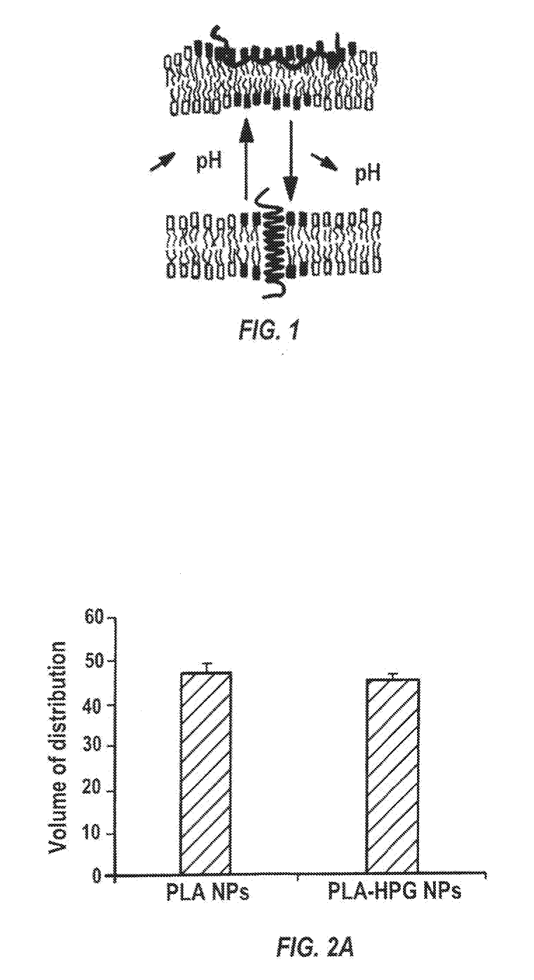

The surface properties of the HPG can be adjusted based on the chemistry of vicinal diols. For example, the surface properties can be tuned to provide stealth particles, i.e., particles that are not cleared by the MPS due to the presence of the hydroxyl groups; adhesive (sticky) particles, i.e., particles that adhere to the surface of tissues, for example, due to the presence of one or more reactive functional groups, such as aldehydes, amines, oxime, or O-substituted oxime that can be prepared from the vicinal hydroxyl moieties; or targeting by the introduction of one or more targeting moieties which can be conjugated directly or indirectly to the vicinal hydroxyl moieties. Indirectly refers to transformation of the hydroxy groups to reactive functional groups that can react with functional groups on molecules to be attached to the surface, such as active agents and/or targeting moieties, etc. A schematic of this tunability is shown in FIG. 1A showing a bioadhesive polymer.

The hyperbranched nature of the polyglycerol allows for a much higher density of hydroxyl groups, reactive functional groups, and/or targeting moieties than obtained with linear polyethylene glycol. For example, the particles can have a density of surface functionality (e.g., hydroxyl groups, reactive functional groups, and/or targeting moieties) of at least about 1, 2, 3, 4, 5, 6, 7, or 8 groups/nm².

The molecular weight of the HPG can vary. For example, in those embodiments wherein the HPG is covalently attached to the materials or polymers that form the core, the molecular weight can vary depending on the molecular weight and/or hydrophobicity of the core materials. The molecular weight of the HPG is generally from about 1,000 to about 1,000,000 Daltons, from about 1,000 to about 500,000 Daltons, from about 1,000 to about 250,000 Daltons, or from about 1,000 to about 100,000 Daltons. In those embodiments wherein the HPG is covalently bound to the core materials, the weight percent of HPG of the copolymer is from about 1% to about 50%, such as about 1, 2, 3, 4, 5, 10, 15, 20, 25, 30, 35, 40, 45 or 50%.

In some embodiments, the HPG is covalently coupled to a hydrophobic material or a more hydrophobic material, such as a polymer. Upon self-assembly, particles are formed containing a core containing the hydrophobic material and a shell or coating of HPG. HPG coupled to the polymer PLA is shown below:

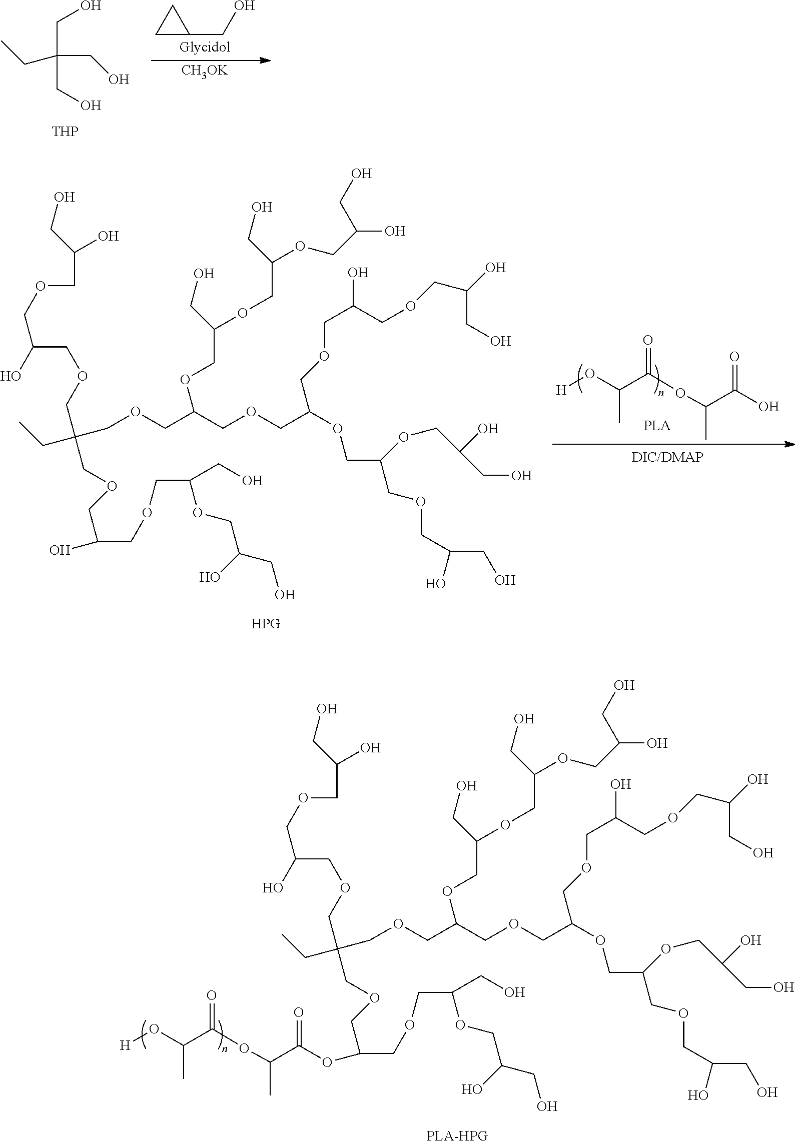

2. Other Polymers for Forming a Shell, Corona or Coating

NPs with bioadhesive coronas are not limited to hyperbranched polyglycerols and their associated aldehydes, but may include other biodegradable polymers and molecules such as peptides formed of amino acids and, oligonucleotides formed of nucleic acids, polysaccharides and fatty acids. These polymers or small molecules, when converted to an aldehyde-terminated form, are adhesive.

Suitable materials for forming bioadhesive functional groups are materials that have aldehydes or the potential to form aldehydes following chemical modification (e.g. sodium periodate (NaIO₄) treatment). These include polymers of saccharides such as dextran, cellulose, and other starches, polymers of or containing serine amino acids or materials with vicinal diol or serine structure (amine and hydroxyl on neighboring carbons), materials with hydroxyl groups, since the hydroxyl groups can be oxidized to aldehydes by catalysts such as Collins reagent, or any polymeric molecule, such as a dendrimer that may be attached with molecules containing aldehydes or has groups may be converted to aldehydes (Gao and Yan, Prog. Polym. Sci. 29:183-275 (2004)).

Below are the vicinal diols (most sugars have vicinal diols) and serine structures, which can be oxidized to aldehydes by NaIO₄ treatment.

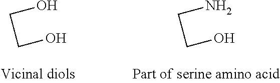

C. Therapeutic, Diagnostic and Prophylactic Agents

The particles may contain one or more types of molecules encapsulated within and/or attached to the surface of the particles. The molecules can be covalently or non-covalently associated with the particles. In some embodiments, the molecules are targeting moieties that are covalently associated with the particles. In particular embodiments, the targeting moieties are covalently bound to the HP coating. For example, when the HP is HPG, targeting moieties can be covalently bound via the hydroxy groups on HPG.

More than conferring stealth properties to the NPs, HPG has a higher density of functional groups available for functionalization compared to the gold standard polyethylene glycol (PEG). Indeed, each HPG chain presents 40 hydroxyl groups on its surface, and each one can be conjugated with functionalizing ligands, when PEG presents only one conjugation site per chain. Therefore the functionalization of HPG will provide a dramatic increase in the number of functionalizing ligands at the surface of the particles, allowing for improved properties.

The targeting moieties can be bound directly to HP or via a coupling agent. In other embodiments, the particles have encapsulated therein one or more therapeutic agents, diagnostic agents, prophylactic agents, and/or nutraceuticals. In some embodiments, the particles contain both targeting agents that are covalently or non-covalently associated with the particles and one or more therapeutic agents, diagnostic agents, prophylactic agents, and/or nutraceuticals that are covalently or non-covalently associated with the particles.

Molecules can be bound to the hydroxy groups on HP before or after particle formation. Representative methodologies for conjugated molecules to the hydroxy groups on HP are described below.

The particles, such as the surface of the particles, can be modified to facilitate targeting or enhance the bioactivity of a bioactive agent therein through the attachment of one or more targeting molecules, one or more sensitizing agents, or a combination thereof.

1. Targeting Moieties

Exemplary target molecules include proteins, peptides, nucleic acids, lipids, saccharides, or polysaccharides, or small molecules that bind to one or more targets associated with an organ, tissue, cell, or extracellular matrix, or specific type of tumor or infected cell. In particular embodiments the targeting moiety is a protein, peptide, antibody or aptamer. The degree of specificity with which the particles are targeted can be modulated through the selection of a targeting molecule with the appropriate affinity and specificity. For example, a targeting moiety can be a polypeptide, such as an antibody that specifically recognizes a tumor marker that is present exclusively or in higher amounts on a malignant cell (e.g., a tumor antigen). Targeting molecules can also include neuropilins and endothelial targeting molecules, integrins, selectins, and adhesion molecules. Targeting molecules can be covalently bound to particles using a variety of methods known in the art. In some embodiments, the targeting moieties are covalently associated with the polymer, preferably via a linker cleaved at the site of delivery.

The nanoparticles can contain one or more polymer conjugates containing end-to-end linkages between the polymer and a targeting element or a detectable label. For example, a modified polymer can be a PLA-HPG-peptide block polymer.

Targeting agents can increase uptake in targeted cells, decrease uptake in non-targeted cells, reduce toxicity to healthy cells, and combinations thereof.

Examples of targeting moieties include peptides such as iRGD, LyP1; small molecule such as folate, aptamers and antibodies or their combinations at various molar ratios.

The targeting element of the nanoparticle can be an antibody or antigen binding fragment thereof. The targeting elements should have an affinity for a cell-surface receptor or cell-surface antigen on the target cells and result in internalization of the particle within the target cell.

The targeting element can specifically recognize and bind to a target molecule specific for a cell type, a tissue type, or an organ. The target molecule can be a cell surface polypeptide, lipid, or glycolipid. The target molecule can be a receptor that is selectively expressed on a specific cell surface, a tissue or an organ. Cell specific markers can be for specific types of cells including, but not limited to stem cells, blood cells, immune cells, muscle cells, nerve cells, cancer cells, virally infected cells, and organ specific cells. The cell markers can be specific for endothelial, ectodermal, or mesenchymal cells. Representative cell specific markers include, but are not limited to cancer specific markers.

Additional targets that can be recognized by the targeting element include VEGF/KDR, Tie2, vascular cell adhesion molecule (VCAM), endoglin and α₅β₃ integrin/vitronectin. The targeting peptides can be covalently associated with the polymer of the outer shell and the covalent association can be mediated by a linker.

Suitable targeting molecules that can be used to direct nanoparticles to cells and tissues of interest are known in the art, see, for example, Ruoslahti, et al. Nat. Rev. Cancer, 2:83-90 (2002), and WO 2015/172149.

A particularly preferred targeting moiety is pHLIP (see, e.g., An, et al., Proc Natl Acad Sci USA., 107(47): 20246-20250 (2010) and Shu, et al., Nature Communications, 6, Article number: 7787 doi:10.1038/ncomms8787 (2015), and references cited therein). pHLIP is a pH-low insertion peptide that can have the sequence (AAEQNPIYWARYADWLFTTPLLLLDLALLVDADEGTG (SEQ ID NO:1)). Another exemplary pHLIP sequence is (GGEQNPIYWARYADWLFTTPLLLLDLALLVDADEGT (SEQ ID NO:2)). In some embodiments, the peptide is or includes a variant having at least 50, 60, 70, 80, 85, 90, 95, 96, 97, 98, 99 sequence identity to SEQ ID NO:1 or 2. The variant can have mutations (i.e., substitution(s), insertion(s), or deletion(s) relative to SEQ ID NO:1 or 2. In some embodiments, the substitution(s) are conservative substitutions. In particular embodiments, 1, 2, 3, 4, 5, 6, 7, 8, 9, or 10 amino acids are deleted from the C-terminus, the N-terminus, or both of SEQ ID NO:1 or 2.

The tumor-targeting ability of pHLIP is thought to be based on its insertion into membrane in response to environmental acidity, a feature common to solid tumor microenvironments. Similarly, pHLIP can detect other pathological acidic microenvironments in vivo, such as those found in inflammation and ischemic myocardium. In addition, the transmembrane (TM) insertion behavior imparts pHLIP with a built-in mechanism for cytoplasmic cargo delivery. Molecules including fluorescent dyes, polar membrane-impermeable peptides (for example, phalloidin and other toxins), and chemotherapy drugs such as paclitaxel have been translocated and released into cells when attached to the inserting carboxy (C) terminus of pHLIP. The examples below show that CED-particles functionalized with pHLIP have increased uptake in tumor cells and accumulate to a higher degree at the periphery of the tumor relative to conventional particles.

2. Sensitizing Agents

Sensitizing agents typically prime the tumor cell or its microenvironment to another bioactive agent. A sensitizing agent can also increase or potentiate the bioactivity of another bioactive agent. For example, in some embodiments, the bioactivity of a bioactive agent has an increased or greater effect at the site of delivery, on the target cells, etc., in the presence of the sensitizing agent relative to bioactive agent alone.

As discussed above, glioblastoma is a particularly deadly condition because glioma stem cells are chemoresistant. To address this phenomenon, therapies that target the signal transduction and biological characteristics of cancer stem cells (CSCs) are being developed and used in combination with conventional chemotherapy and radiotherapy in an effort to reduce the recurrence and improve treatment.

The two strategies are (a) chemotherapeutic regimens that specifically drive CSCs toward cell death and (b) those that promote the differentiation of CSCs, thereby depleting the tumor reservoir (Daniele, et al., Cell Death and Disease, 5, e1539; doi:10.1038/cddis.2014.487 (2014)). Thus, the sensitizing agent can be one that drives cancer stem cells, such as glioma stem cells, toward cell death; promotes the differentiation of cancer stem cells, such as glioma stem cells; or a combination thereof.

Extracellular purines, particularly adenosine triphosphate, have been implicated in the regulation of CSC formation. Studies showed that stimulation of purinergic receptors for adenosine (adenosine receptors (AR)), particularly the A₁ and A_{2B} receptors, has a prominent anti-proliferative/pro-apoptotic effect on the CSCs (Daniele, et al., Cell Death and Disease, 5, e1539; doi:10.1038/cddis.2014.487 (2014)). It has also been shown that glioblastoma grows more vigorously in A1 adenosine receptor (A₁ AR)-deficient mice indicating an anti-tumorogenic action of adenosine when stimulating its A₁ receptor (Synowitz, et al., Cancer Res., 66(17):8550-7 (2006)). It was further demonstrated that this anti-tumorogenic effect was mediated by microglia cells. In Daniele, et al., an A₁ AR agonist was found to promote the differentiation of CSCs toward a glial phenotype, and both A₁ and A_{2B} AR agonists sensitized CSCs to the genotoxic activity of temozolomide (TMZ) and prolonged its effects. It is believed that A_{2B} AR potentiated the pro-apoptotic effects of TMZ and that A₁ AR drove cells toward a differentiated phenotype that is more sensitive to TMZ. Thus in some embodiments, the sensitizing agent (i) promotes cell apoptosis directly or indirectly by increasing the apoptotic effect of a second bioactive agent such as a chemotherapeutic drug; or (ii) induces differentiation of cells toward a phenotype more sensitive to a second bioactive agent, or a combination thereof.

In some embodiments, the sensitizing agent is an adenosine receptor agonist. Exemplary agonists include, but are not limited to, (2R,3R,4S,5R)-2-(6-amino-9H-purin-9-yl)-5-(hydroxymethyl)oxolane-3,4-diol (adenosine), 4-[2-[[6-Amino-9-(N-ethyl-3-D-ribofuranuronamidosyl)-9H-purin-2-yl]amino]ethyl]benzenepropanoic acid hydrochloride (CGS 21680), N6-cyclo-hexyladenosine (CHA), 2-Chloro-N-cyclopentyladenosine (CCPA), 2-Chloro-N-cyclopentyl-2′-methyladenosine (2′-MeCCPA), N-Cyclopentyladenosine (CPA), 3-[4-[2-[[6-amino-9-[(2R,3R,4S,5S)-5-(ethylcarbamoyl)-3,4-dihydroxy-oxolan-2-yl]purin-2-yl]amino]ethyl]phenyl]propanoic acid (CGS21680), 2-(1-Hexynyl)-N-methyladenosine (HEMADO), 2-chloro-N6-(3-iodobenzyl)adenosine-5′-N-methylcarboxamide (Cl-IB-MECA), 1-[2-Chloro-6-[[(3-iodophenyl)methyl]amino]-9H-purin-9-yl]-1-deoxy-N-methyl-β-D-ribofuranuronamide (2-Cl-IB-MECA), 1-Deoxy-1-[6-[[(3-iodophenyl)methyl]amino]-9H-purin-9-yl]-N-methyl-3-D-ribofuranuronamide (IB-MECA), [2-[6-Amino-3,5-dicyano-4-[4-(cyclopropylmethoxy)-phenyl]pyridin-2-ylsulfanyl]acetamide] (BAY606583), 5′-N-Ethylcarboxamidoadenosine (NECA), and N-Cyclohexyl-2′-O-methyladenosine (SDZ WAG 994). Certain miRNAs and anti-microRNAs can act as sensitizing factors, such as anti-mir-2.

In some embodiments, the adenosine receptor agonist is adenosine:

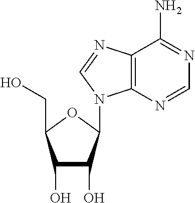

which has been shown to sensitive glioma stem cells (GSCs) (Daniele S, et al., Cell Death Dis, 5:e1539 (2014)) and modulate the tumor microenvironment (Synowitz, et al. Cancer Res., 66:8550-8557 (2006)).

3. Active Agents

Agents to be delivered include therapeutic, nutritional, diagnostic, and prophylactic compounds. Proteins, peptides, carbohydrates, polysaccharides, nucleic acid molecules, and organic molecules, as well as diagnostic agents, can be delivered. The preferred materials to be incorporated are drugs and imaging agents. Therapeutic agents include antibiotics, antivirals, anti-parasites (helminths, protozoans), anti-cancer (referred to herein as “chemotherapeutics”, including cytotoxic drugs such as doxorubicin, cyclosporine, mitomycin C, cisplatin and carboplatin, BCNU, SFU, methotrexate, adriamycin, camptothecin, epothilones A-F, and taxol), antibodies and bioactive fragments thereof (including humanized, single chain, and chimeric antibodies), antigen and vaccine formulations, peptide drugs, anti-inflammatories, nutraceuticals such as vitamins, and oligonucleotide drugs (including DNA, RNAs including mRNAs, antisense, siRNA, miRNA, anti-miRNA, piRNA, aptamers, ribozymes, external guide sequences for ribonuclease P, and triplex forming agents such as tcPNAs). In some embodiments, the active agent is a vector, plasmid, or other polynucleotide encoding an oligonucleotide such as those discussed above.

Particularly preferred drugs to be delivered include anti-angiogenic agents, antiproliferative and chemotherapeutic agents such as rampamycin. Incorporated into particles, these agents may be used to treat cancer or eye diseases, or prevent restenosis following administration into the blood vessels.

Representative classes of diagnostic materials include paramagnetic molecules, fluorescent compounds, magnetic molecules, and radionuclides. Exemplary materials include, but are not limited to, metal oxides, such as iron oxide, metallic particles, such as gold particles, etc. Biomarkers can also be conjugated to the surface for diagnostic applications.

One or more active agents may be formulated alone or with excipients or encapsulated on, in or incorporated into the particles. Active agents include therapeutic, prophylactic, neutraceutical and diagnostic agents. Any suitable agent may be used. These include organic compounds, inorganic compounds, proteins, polysaccharides, nucleic acids or other materials that can be incorporated using standard techniques.

Active agents include synthetic and natural proteins (including enzymes, peptide-hormones, receptors, growth factors, antibodies, signaling molecules), and synthetic and natural nucleic acids (including RNA, DNA, anti-sense RNA, triplex DNA, inhibitory RNA (RNAi), and oligonucleotides), and biologically active portions thereof. Suitable active agents have a size greater than about 1,000 Da for small peptides and polypeptides, more typically at least about 5,000 Da and often 10,000 Da or more for proteins. Nucleic acids are more typically listed in terms of base pairs or bases (collectively “bp”). Nucleic acids with lengths above about 10 bp are typically used in the present method. More typically, useful lengths of nucleic acids for probing or therapeutic use will be in the range from about 20 bp (probes; inhibitory RNAs, etc.) to tens of thousands of bp for genes and vectors. The active agents may also be hydrophilic molecules, preferably having a low molecular weight.

Examples of useful proteins include hormones such as insulin and growth hormones including somatomedins. Examples of useful drugs include neurotransmitters such as L-DOPA, antihypertensives or saluretics such as Metolazone from Searle Pharmaceuticals, carbonic anhydrase inhibitors such as Acetazolamide from Lederle Pharmaceuticals, insulin like drugs such as glyburide, a blood glucose lowering drug of the sulfonylurea class, synthetic hormones such as Android F from Brown Pharmaceuticals and Testred® (methyltestosterone) from ICN Pharmaceuticals.

Representative anti-cancer agents include, but are not limited to, alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and decetaxel, epothilones A-F, and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), topoisomerase inhibitors (including camptothecins such as camptothecin, irinotecan, and topotecan as well as derivatives of epipodophyllotoxins such as amsacrine, etoposide, etoposide phosphate, and teniposide), and combinations thereof. Other suitable anti-cancer agents include angiogenesis inhibitors including antibodies to vascular endothelial growth factor (VEGF) such as bevacizumab (AVASTIN®), other anti-VEGF compounds; thalidomide (THALOMID®) and derivatives thereof such as lenalidomide (REVLIMID®); endostatin; angiostatin; receptor tyrosine kinase (RTK) inhibitors such as sunitinib (SUTENT®); tyrosine kinase inhibitors such as sorafenib (Nexavar®), erlotinib (Tarceva®), pazopanib, axitinib, and lapatinib; transforming growth factor-α or transforming growth factor-β inhibitors, and antibodies to the epidermal growth factor receptor such as panitumumab (VECTIBIX®) and cetuximab (ERBITUX®), as well as some of the new drugs such as Ipilimumab and nivolumab, etc.

Under the Biopharmaceutical Classification System (BCS), drugs can belong to four classes: class I (high permeability, high solubility), class II (high permeability, low solubility), class III (low permeability, high solubility) or class IV (low permeability, low solubility). Suitable active agents also include poorly soluble compounds; such as drugs that are classified as class II or class IV compounds using the BCS. Examples of class II compounds include: acyclovir, nifedipine, danazol, ketoconazole, mefenamic acid, nisoldipine, nicardipine, felodipine, atovaquone, griseofulvin, troglitazone glibenclamide and carbamazepine. Examples of class IV compounds include: chlorothiazide, furosemide, tobramycin, cefuroxmine, and paclitaxel.

For imaging, radioactive materials such as Technetium99 (^{99m}Tc) or magnetic materials such as Fe₂O₃ could be used. Examples of other materials include gases or gas emitting compounds, which are radioopaque. The most common imaging agents for brain tumors include iron oxide and gadolinium.

Alternatively, the biodegradable polymers may encapsulate cellular materials, such as for example, cellular materials to be delivered to antigen presenting cells as described below to induce immunological responses.

In the preferred embodiment, drugs that have already been approved for clinical use are screened for delivery and efficacy in treatment of the CNS, especially brain tumors such as glioblastomas.

Representative therapeutic agents include vascular endothelial growth factor (“VEGF”) or VEGF receptor inhibitors such as bevacizumab, alkylating agents such as temozolomide or BCNU (carmustine), and other antineoplastics such as procarbazine. Preferred compounds include Carmustine (BCNU), temozolomide, taxols such as paclitaxel, camptothecine (CPT), and dithiazanine iodide (DI). The particles can also be used to deliver short acting radioactive compounds.

Other preferred compounds include DNA repair inhibitors, radiosensitizers, and replication checkpoint modulators. In some embodiments, the replication modulator can, for example, manipulate replication progression and/or replication forks. For example, the ATR-Chk1 cell cycle checkpoint pathway has numerous roles in protecting cells from DNA damage and stalled replication. One of the most prominent is the control of the cell cycle and the prevention of premature entry into mitosis (Thompson and Eastman, Br J Clin Pharmacol., 76(3): 358-369 (2013), Smith, et al., Adv Cancer Res., 108:73-112 (2010)). However, Chk1 also contributes to the stabilization of stalled replication forks, the control of replication origin firing and replication fork progression, and homologous recombination. Other replication modulators are DNA polymerase alpha (also known as Pol α), which is an enzyme complex found in eukaryotes that is involved in initiation of DNA replication (Alama, et al., Exp Cell Res., 206(2):318-22 (1993)), or Hsp90 (heat shock protein 90), which is a chaperone protein that assists other proteins to fold properly, stabilizes proteins against heat stress, and aids in protein degradation (Gomez-Monterrey, et al., Recent Pat Anticancer Drug Discov., 7(3):313-36 (2012)).

In some embodiments, the active agent is a Chk1 or ATR pathway inhibitor, a DNA polymerase alpha inhibitor, or an HSP90 inhibitor. The inhibitor can be a functional nucleic acid, for example siRNA, miRNA, aptamers, ribozymes, triplex forming molecules, RNAi, or external guide sequences that targets Chk1, ATR, or another molecule in the ATR-Chk1 cell cycle checkpoint pathway; DNA Pol α; or HSP90, and reduces expression of ATR, Chk1, DNA Pol α, or HSP90.

Preferably, the inhibitor is a small molecule. For example, the potentiating factor can be a small molecule inhibitor of ATR-Chk1 Cell Cycle Checkpoint Pathway Inhibitor. Such inhibitors are known in the art, and many have been tested in clinical trials for the treatment of cancer. Exemplary Chk1 inhibitors include, but are not limited to, AZD7762, SCH900776/MK-8776, IC83/LY2603618, LY2606368, GDC-0425, PF-00477736, XL844, CEP-3891, SAR-020106, CCT-244747, Arry-575 (Thompson and Eastman, Br J Clin Pharmacol., 76(3): 358-369 (2013)), and SB218075. Exemplary ATR pathway inhibitors include, but are not limited to Schisandrin B, NU6027, NVP-BEZ235, VE-821, VE-822 (VX-970), AZ20, AZD6738, MIRIN, KU5593, VE-821, NU7441, LCA, and L189 (Weber and Ryan, Pharmacology & Therapeutics, 149:124-138 (2015)).

In some embodiments, the active agent is a DNA Pol α inhibitor, such as aphidicolin.

In some embodiments, the active agent is a heat shock protein 90 inhibitor (HSP90i) such as STA-9090 (ganetespib). Other HSP90 inhibitors are known in the art and include, but are not limited to, benzoquinone ansamycin antibiotics such as geldanamycin (GA); 17-AAG (17-Allylamino-17-demethoxy-geldanamycin); 17-DMAG (17-dimethylaminoethylamino-17-demethoxy-geldanamycin) (Alvespimycin); IPI-504 (Retaspimycin); and AUY922 (Tatokoro, et al., EXCLI J., 14:48-58 (2015)).

Prophylactics can include compounds alleviating swelling, reducing radiation damage, and anti-inflammatories.

Diagnostic agents can be radioactive, magnetic, or x-ray or ultrasound-detectable.

Typical loadings of the particles, based on weight, are in the range of, for example, 0.1 to 20%, with more typical values between 1-10%

4. Sheddable Polyethylene Glycol (PEG) Coatings

HPG-coated particles can be modified by covalently attaching PEG to the surface. This can be achieved by converting the vicinyl diol groups to aldehydes and then reacting the aldehydes with functional groups on PEG, such as aliphatic amines, aromatic amines, hydrazines and thiols. The linker has end groups such as aliphatic amines, aromatic amines, hydrazines, thiols and O-substituted oxyamines. The bond inserted in the linker can be disulfide, orthoester and peptides sensitive to proteases.

PEG with a functional group or a linker can form a bond with aldehyde on PLA-HPG-CHO and reversed the bioadhesive state of PLA-HPG-CHO to stealth state. This bond or the linker is labile to pH change or high concentration of peptides, proteins and other biomolecules. After administration systematically or locally, the bond attaching the PEG to PLA-HPG-CHO can be reversed or cleaved to release the PEG in response to environment, and expose the bioadhesive PLA-HPG-CHO particles to the environment. Subsequently, the particles will interact with the tissue and attach the particles to the tissues or extracellular materials such as proteins. The environment can be acidic environment in tumors, reducing environment in tumors, protein rich environment in tissues.

### III. Method of Making Polymeric Particles

A. Particle Properties

The particles may have any zeta potential. The particles can have a zeta potential from −300 mV to +300 mV, −100 mV to +100 mV, from −50 mV to +50 mV, from −40 mV to +40 mV, from −30 mV to +30 mV, from −20 mV to +20 mV, from −10 mV to +10 mV, or from −5 mV to +5 mV. The particles can have a negative or positive zeta potential. In some embodiments the particles have a substantially neutral zeta potential, i.e. the zeta potential is approximately 0 mV. In preferred embodiments the particles have a zeta potential of approximately −30 to about 30 mV, preferably from about −20 to about 20 mV, more preferably from about −10 to about 10 mV.

The particles may have any diameter. The particles can have an average diameter of between about 1 nm and about 1000 microns, about 1 nm and about 100 microns, about 1 nm and about 10 microns, about 1 nm and about 1000 nm, about 1 nm and about 500 nm, about 1 nm and about 250 nm, or about 1 nm and about 100 nm. In preferred embodiments, the particle is a nanoparticle having a diameter from about 25 nm to about 250 nm, more particularly 40 to 200 nm.

For administration to the brain, particularly when delivered locally by injection, infusion, or convection enhanced delivery, the nanoparticles have a diameter from about 25 nm to about 120 nm, from about 40 nm to about 100 nm, or from about 60 nm to about 90 nm, or from about 35 nm to about 60 nm. In some applications, particularly those in non-tumor regions of the brain, the particles are larger than those for administration into the tumor.

Particles size typically is based on a population, wherein 60, 70, 80, 85, 90, or 95% of the population has the desired size range.

The polydispersity can be from about 0.01 to 0.30, or from about 0.01 to about 0.25, or from about 0.01 to about 0.20, or from about 0.01 to about 0.15, or from about 0.01 to about 0.10.

B. Manufacturing Particles

Methods of making polymeric particles are known in the art. Common microencapsulation techniques include, but are not limited to, spray drying, interfacial polymerization, hot melt encapsulation, phase separation encapsulation (spontaneous emulsion microencapsulation, solvent evaporation microencapsulation, and solvent removal microencapsulation), nano-precipitation, coacervation, low temperature microsphere formation, and phase inversion nanoencapsulation (PIN). A brief summary of these methods is presented below.

In some embodiments, the particles are prepared using an emulsion-based technique. In particular embodiments, the particles are prepared using a double emulsion solvent evaporation technique. For example, amphiphilic material and hydrophobic cationic material are dissolved in a suitable organic solvent, such as methylene chloride or dichloromethane (DCM), with or without a therapeutic agent. The active agent, for example a nucleic acid, such as siRNA or a mimic thereof, is reconstituted in purified water, such as HyPure™ molecular biology grade water (Hyclone Laboratories, Inc., Logan, Utah). The siRNA solution is added dropwise to the solution of the amphiphilic material and the hydrophobic cationic material and emulsified to form a first emulsion. The emulsion is added to an aqueous solution of surfactant, such as PVA, to form a double emulsion. The final emulsion is added to water and stirred for an extended period of time (e.g., 3 hours) to allow the organic solvent to evaporate and the particles to harden. Residual organic solvent and/or unencapsulated molecules are removed by washing.

In some embodiments, a partially water-miscible organic solvent is preferred. Partially water-miscible solvents such as benzyl alcohol, butyl lactate, and ethyl acetate (EA), allow nanoparticle formulation through an emulsion-diffusion mechanism and are able to produce smaller nanoparticles than water-immiscible solvents such as DCM. In some embodiments, the solvent is a “generally regarded as safe” (GRAS) solvent. TEA is a preferred partially water-miscible organic solvent for clinical applications due to its low toxicity.

Other emulsion emulsion-based procedures are described below.

1. Phase Separation Microencapsulation

In phase separation microencapsulation techniques, a polymer solution is stirred, optionally in the presence of one or more active agents to be encapsulated. While continuing to uniformly suspend the material through stirring, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the active agent(s) in a droplet with an outer polymer shell.

2. Spontaneous Emulsion Microencapsulation

Spontaneous emulsification involves solidifying emulsified liquid polymer droplets formed above by changing temperature, evaporating solvent, or adding chemical cross-linking agents. The physical and chemical properties of the encapsulant, as well as the properties of the one or more active agents optionally incorporated into the nascent particles, dictates suitable methods of encapsulation. Factors such as hydrophobicity, molecular weight, chemical stability, and theimal stability affect encapsulation.

3. Solvent Evaporation Microencapsulation

Methods for forming microspheres using solvent evaporation techniques are described in E. Mathiowitz et al., J. Scanning Microscopy, 4:329 (1990); L. R. Beck et al., Fertil. Steril., 31:545 (1979); L. R. Beck et al, Am. J Obstet. Gynecol., 135(3) (1979); S. Benita et al., J. Pharm. Sci., 73:1721 (1984); and U.S. Pat. No. 3,960,757 to Morishita et al. The polymer is dissolved in a volatile organic solvent, such as methylene chloride. One or more active agents to be incorporated are optionally added to the solution, and the mixture is suspended in an aqueous solution that contains a surface active agent such as poly(vinyl alcohol). The resulting emulsion is stirred until most of the organic solvent evaporated, leaving solid microparticles/nanoparticles. This method is useful for relatively stable polymers like polyesters and polystyrene.

4. Phase Inversion Nanoencapsulation (PIN)

Nanoparticles can also be formed using the phase inversion nanoencapsulation (PIN) method, wherein a polymer is dissolved in a “good” solvent, fine particles of a substance to be incorporated, such as a drug, are mixed or dissolved in the polymer solution, and the mixture is poured into a strong non solvent for the polymer, to spontaneously produce, under favorable conditions, polymeric microspheres, wherein the polymer is either coated with the particles or the particles are dispersed in the polymer. See, e.g., U.S. Pat. No. 6,143,211 to Mathiowitz, et al. The method can be used to produce monodisperse populations of nanoparticles and microparticles in a wide range of sizes, including, for example, about 100 nanometers to about 10 microns.

5. Microfluidics

Nanoparticles can be prepared using microfluidic devices. A polymeric material is mixed with a drug or drug combinations in a water miscible organic solvent. The water miscible organic solvent can be one or more of the following: acetone, ethanol, methanol, isopropyl alcohol, acetonitrile and Dimethyl sulfoxide (DMSO). The resulting mixture solution is then added to an aqueous solution to yield nanoparticle solution. The targeted peptides or fluorophores or drugs may be associated with the surface of, encapsulated within, surrounded by, and/or distributed throughout the polymeric matrix of the particles.

6. Nanoprecipitation

In nanoprecipitation, the polymer and active agent (e.g., nucleic acids) are co-dissolved in a selected, water-miscible solvent, for example DMSO, acetone, ethanol, acetone, etc. In a preferred embodiment, active agent and polymer are dissolved in DMSO. The solvent containing the polymer and active agent is then drop-wise added to an excess volume of stirring aqueous phase containing a stabilizer (e.g., poloxamer, Pluronic®, and other stabilizers known in the art). Particles are formed and precipitated during solvent evaporation. To reduce the loss of polymer, the viscosity of the aqueous phase can be increased by using a higher concentration of the stabilizer or other thickening agents such as glycerol and others known in the art. Lastly, the entire dispersed system is centrifuged, and the nucleic acid-loaded polymer nanoparticles are collected and optionally filtered. Nanoprecipitation-based techniques are discussed in, for example, U.S. Pat. No. 5,118,528.

Advantages to nanoprecipitation include: the method can significantly increase the encapsulation efficiency of drugs that are polar yet water-insoluble, compared to single or double emulsion methods (Alshamsan, Saudi Pharmaceutical Journal, 22(3):219-222 (2014)). No emulsification or high shear force step (e.g., sonication or high-speed homogenization) is involved in nanoprecipitation, therefore preserving the conformation of nucleic acids. Nanoprecipitation relies on the differences in the interfacial tension between the solvent and the nonsolvent, rather than shear stress, to produce nanoparticles. Hydrophobicity of the drug will retain it in the instantly-precipitating nanoparticles; the un-precipitated polymer due to equilibrium is “lost” and not in the precipitated nanoparticle form.

C. HP Conjugates or Coatings

Hyperbranched polymers including, but not limited to, hyperbranched polyglycerol (HPG), can be covalently bound to one or more materials, such as a polymer, that form the core of the particles using methodologies known in the art. For example, an HP such as HPG can be covalently coupled to a polymer having carboxylic acid groups, such as PLA, PGA, or PLGA using DIC/DMAP.

The HPG can be initiated from hydroxyl, amine, and carboxylate terminated molecules, such as an alcohol with one or multiple long hydrophobic tail. In another example, the HP, such as HPG, can be initiated from special functionalized initiators to facilitate the conjugation to more materials. These special initiators include disulfide (Yeh et al., Langmuir. 24(9):4907-16(2008)).

The HPG can be functionalized to introduce one or more reactive functional groups that alter the surface properties of the particles. The surface of the particles can further be modified with one or more targeting moieties or covalently bound to an HP such as HPG via a coupling agent or spacer in organic such as dichloromethane (DCM), dimethylformamide (DMF), dimethyl sulfoxide (DMSO), tetrahydrofuran (THF), diisopropylcarbodiimide (DIC), 4-(N,N-dimethylamino)pyridine (DMAP), dicyclohexylcarbodiimide (DCC), DIC/DMAP, DCC/DMAP, Acylchloride/pyridine. In some embodiments, the polymer is functionalized/modified before nanoparticle formation. Alternatively, the targeting moieties may be attached to NPs after the synthesis of NPs in aqueous solution (or other protic solution such as alcohol). As discussed in more detail below, HPG coated NPs can be transformed to aldehyde terminated NPs by NaIO₄ treatment (or carboxylic acid terminated by NaIO₄ treatment followed by sodium chlorite treatment) so the targeting moieties may be directly covalently attached to NPs via aldehyde (or carboxylic acid) groups on NPs and functional groups (amine, hydrazine, amino-oxy and their derivatives) on the targeting moieties or indirectly attached to the NPs via coupling agents or spacers (such as amino-oxy modified biotin and cysteine).

Certain properties of the PLA-HPG conjugate are important for the observed effects thereof. Because high molecular weight HPG has better resistance to non-specific adsorption to biomolecules, the low molecular weight components can be removed from the synthesized HPG by multiple solvent precipitations and dialysis.

In the preferred embodiment, a polyhydroxy acid such as PLA is selected as the hydrophobic core material because it is biodegradable, has a long history of clinical use, and is the major component of a NP system that is advancing in clinical trials. To covalently attach the PLA to HPG, the previous approach was to first functionalize the HPG with an amine and then conjugate the carboxylic group on PLA to the amine. This approach is efficient but cannot be used to make HPG as surface coatings since any amines that do not react with PLA will lead to a net positive charge on the neutral HPG surface and reduce the ability of HPG to resist adsorption of other molecules on the surface. To avoid this, a one-step esterification between PLA and HPG can be employed, which maintains the charge neutral state of the HPG.

Targeting molecules or agents to be encapsulated or delivered may be associated with the surface of, encapsulated within, surrounded by, and/or distributed throughout the polymeric matrix of the particles.

D. Functionalizing Nanoparticles

Representative methodologies for conjugated molecules to the hydroxy groups on HP are provided. One useful protocol involves the “activation” of hydroxyl groups with carbonyldiimidazole (CDI) in aprotic solvents such as DMSO, acetone, or THF. CDI forms an imidazolyl carbamate complex with the hydroxyl group which may be displaced by binding the free amino group of a ligand such as a protein. The reaction is an N-nucleophilic substitution and results in a stable N-alkylcarbamate linkage of the ligand to the polymer. The “coupling” of the ligand to the “activated” polymer matrix is maximal in the pH range of 9-10 and normally requires at least 24 hrs. The resulting ligand-polymer complex is stable and resists hydrolysis for extended periods of time.

Another coupling method involves the use of 1-ethyl-3-(3-dimethylaminopropyl) carbodiimide (EDAC) or “water-soluble CDI” in conjunction with N-hydroxylsulfosuccinimide (sulfo NHS) to couple the exposed carboxylic groups of polymers to the free amino groups of ligands in a totally aqueous environment at the physiological pH of 7.0. Briefly, EDAC and sulfo-NHS form an activated ester with the carboxylic acid groups of the polymer which react with the amine end of a ligand to form a peptide bond. The resulting peptide bond is resistant to hydrolysis. The use of sulfo-NHS in the reaction increases the efficiency of the EDAC coupling by a factor of ten-fold and provides for exceptionally gentle conditions that ensure the viability of the ligand-polymer complex.

By using either of these protocols it is possible to “activate” almost all polymers containing either hydroxyl or carboxyl groups in a suitable solvent system that will not dissolve the polymer matrix.

A useful coupling procedure for attaching ligands with free hydroxyl and carboxyl groups to polymers involves the use of the cross-linking agent, divinylsulfone. This method would be useful for attaching sugars or other hydroxylic compounds with bioadhesive properties to hydroxylic matrices. Briefly, the activation involves the reaction of divinylsulfone to the hydroxyl groups of the polymer, forming the vinylsulfonyl ethyl ether of the polymer. The vinyl groups will couple to alcohols, phenols and even amines. Activation and coupling take place at pH 11. The linkage is stable in the pH range from 1-8 and is suitable for transit through the intestine.

Alternatively, the hydroxyl groups can be converted to reactive functional group that can react with a reactive functional group on the molecule to be attached. For example, the hydroxyl groups on HP can be converted to aldehydes, amines, or O-substituted oximes, which can react with reactive functional groups on molecules to be attached. Such transformations can be done before or after particle formation.

Any suitable coupling method known to those skilled in the art for the coupling of ligands and polymers with double bonds, including the use of UV crosslinking, may be used for attachment of molecules to the polymer.

Coupling is preferably by covalent binding but it may also be indirect, for example, through a linker bound to the polymer or through an interaction between two molecules such as strepavidin and biotin. It may also be by electrostatic attraction by dip-coating.

The most efficient reaction between —OH and —COOH is to use coupling reagents: DCC/DMAP and DIC/DMAP or activate the —COOH to —COCl and then react with —OH in the presence of pyridine.

The coupling methods can be done before or after particle formation.

1. Exemplary Methods of Functionalization

a. Functionalization of Particles by Synthesizing Functionalized Polymers Before Forming the Particles

The functionalization of polymer-HP can be obtained by coupling hydroxyl groups of the HP with a carboxylic group on the ligand. In the Examples below, PLA-HPG polymer was functionalized with the small molecule adenosine, under a carboxylic modified form (2′,3′-isopropylideneadenosine-5′-carboxylic acid). PLA-HPG can be added to 2′,3′-isopropylidene adenosine-5′-carboxylic acid and dissolved in anhydrous DMF. The solution can be dried with a molecular sieve with DIC and DMAP added to the solution. To purify the polymer, the solution can be added into cold diethyl ether to precipitate the polymer. The polymer precipitate can be collected and dissolved in DCM/TFA mixture (DCM:TFA=2:1) and the reaction shaken at room temperature. The resulting solution can be added into cold diethyl ether and the polymer collected by centrifugation. The polymer can be further purified by redissolving in DCM and precipitating in diethyl ether. To confirm conjugation of Ad to PLA-HPG, the polymers can be dissolved in DMSO-d6 and analyzed by ¹H NMR. The PLA-HPG-Adenosine polymer can be then used to form PLA-HPG-Adenosine nanoparticles using, for example, an emulsion solvent evaporation technique.

b. Functionalization of Pre-Formed Polymer-HPG Particles

Functionalization of pre-formed polymer-HP particles can be carried out by a Schiff base reaction. The hydroxyl groups of the HP at the surface of the particles are first turned into aldehyde groups and further react with an amine group on the ligand. In the Examples below, PLA-HPG nanoparticles were functionalized with a pH-sensitive peptide, pHLIP. The particles can be first prepared using, for example, an emulsion solvent evaporation technique. They can then be rendered “sticky” by converting the alcohol or hydroxyl groups of the HPG into aldehydes using NaIO₄ as introduced above. Following this treatment, the NaIO₄ can be quenched using Na₂SO₃ and the particles can be incubated with ligand to induce a Schiff base reaction between an amino-oxy group on the ligand (e.g., N-terminus of a peptide) and the aldehyde groups. After incubation, the unreacted ligand is washed by centrifugation, and the remaining reactive aldehyde groups on the HPG can be blocked by hydroxyl amine (HONH₂).

As discussed above, “sticky” particles with bioadhesive coronas are not limited to hyperbranched polyglycerols and their associated aldehydes, but may include other biodegradable polymers and molecules such as peptides formed of amino acids and, oligonucleotides formed of nucleic acids, polysaccharides and fatty acids. These polymers or small molecules, when converted to an aldehyde-terminated form, can also be reacted with an amine group on the ligand.

Both conjugation strategies before or after formation of the NPs involve simple and cheap reactions that can be applied to any molecule presenting either a carboxylic group (functionalization before formation of the NPs) or a primary amine group (functionalization after formation of the NPs). This versatility represents the technical advantage of the functionalization of the disclosed platform. As discussed in more detail in the Examples below, functionalized particles formed according to each of the above methods were successfully administered to tumor bearing rats by CED, showing survival improvement in the case of PLA-HPG-Ad nanoparticles loaded with camptothecin (due to the sensitization of the tumor cells by adenosine to the camptothecin activity), and nanoparticle accumulation at the tumor site in the case of PLA-HPG-pHLIP NPs (thanks to the pH-sensitivity of pHLIP inducing accumulation in the acidic area of the tumor).

In some embodiments, the nanoparticles are functionalized with two or more moieties. This can be accomplished by any suitable means, including, for example, either one of the above strategies individually, or both in series. For example, in some embodiments, the functionalization of polymer-HP is obtained by coupling hydroxyl groups on the HP with a carboxylic group on two or more different ligands to create two or more different populations of polymer-HP-ligand that can be mixed together to form particles displaying the two or more different ligands. The two or more different ligands can be reacted with the polymer-HP in the same reaction (e.g., using a pool of two or more ligands) or two or more separate reactions. In some embodiments, functionalization of pre-formed polymer-HP particles can be carried out by a Schiff base reaction, wherein hydroxyl groups of the HP at the surface of the particles are first turned into aldehyde groups and further react with an amine group on two or more ligands.

The amount of ligand displayed on the surface of the particles can also be controlled by, for example, forming the particles with a combination of pre-formed polymer-HP-ligand and unfunctionalized polymer-HP. A higher ratio of polymer-HP-ligand to polymer-HP results in a relatively higher display of the ligand on the surface of the particle, and a lower ratio of polymer-HP-ligand to polymer-HP results in a relatively lower display of the ligand on the surface of the particle. In some embodiments, particles formed of a mixture of pre-formed polymer-HP-ligand and unfunctionalized polymer-HP are subjected to a further step that functionalizes the unfunctionalized polymer-HP by, for example, a Schiff base reaction as discussed above. The same principles can be applied to tune the relative display of two, three or more moieties.

E. Selection of Brain Penetrating Particles

To synthesize standard nanoparticles, following the solvent evaporation phase, the nanoparticle solution is typically subjected to centrifugation speeds suitable to collect particles in the range of about 120-200 nm (e.g., 11,500×g for 15 min,×3) and the pellet is collected. To synthesize brain-penetrating nanoparticles, following a solvent evaporation phase, the nanoparticle solution can first centrifuged at slightly lower speed (e.g., 8,000×g for 10 min) to pellet the large particles. The supernatant can be decanted and brain-penetrating nanoparticles can be collected through high-speed ultracentrifugation to collect the small particles remaining in the supernatant (e.g., 100,000×g for 30 min,×2). For example, in some embodiments, brain penetrating particles having a size of about 60 nm to about 90 nm or 100 nm are prepared by subjecting a polymer/agent solution to single-emulsion solvent evaporation to form a nanoparticle solution; centrifuging the nanoparticle solution at a slow speed to foiui a first pellet and a first supernatant; discarding the first pellet and centrifuging the first supernatant at high speed to form a second pellet, and suspending the second pellet in a pharmaceutically acceptable carrier.

### IV. Particle Formulations

A. Aggregation Reducing Additives

Although not mandatory, lyophilization can be used to stabilize nanoparticles for long-term storage. The additives can be added to reduce aggregation of the particles during before, during, and after storage. Additives include trehalose, other sugars, and other aggregation-reducing materials that can be added to any solution including particles, for example, a resuspension solution and/or a pharmaceutical composition for administration to subject in need thereof. In a particular preferred embodiment, the additive is a sugar such as the FDA-approved disaccharide trehalose. Other sugars include glucose, sucrose and lactose. In preferred embodiments, when utilized as an additive the sugar or other agent is not covalently conjugated to the particle. The additive can be present before lyophilization, after lyophilization or both. In some embodiments, the additive is present even if the particles are never lyophilized, or only added after resuspension from lyophilization. In some embodiments, the additive is present in the pharmaceutical composition administered to a subject in need thereof.

Typically, the weight ratio of sugar to nanoparticles is between 10-50%. For example, in a particular embodiment, the additive is a sugar at a ratio of 0.5:1 (trehalose, mannitol, glucose or sucrose:nanoparticles).

B. Pharmaceutical Compositions

The particles can be formulated with appropriate pharmaceutically acceptable carriers into pharmaceutical compositions for administration to an individual in need thereof. The formulations can be administered enterally (e.g., oral) or parenterally (e.g., by injection or infusion).

The particles can be formulated for parenteral administration. “Parenteral administration”, as used herein, means administration by any method other than through the digestive tract or non-invasive topical or regional routes. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

In preferred embodiments, the particles are administered locally to the central nervous system, particularly the brain, by injection or infusion. In more specific embodiments, the particles are administered to the central nervous system, particularly the brain, by convection enhanced delivery (CED).

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol mono stearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta-alanine, sodium N-laurylβ-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

Enteral formulations are prepared using pharmaceutically acceptable carriers. As generally used herein “carrier” includes, but is not limited to, diluents, preservatives, binders, lubricants, disintegrators, swelling agents, fillers, stabilizers, and combinations thereof. Polymers used in the dosage form include hydrophobic or hydrophilic polymers and pH dependent or independent polymers. Preferred hydrophobic and hydrophilic polymers include, but are not limited to, hydroxypropyl methylcellulose, hydroxypropyl cellulose, hydroxyethyl cellulose, carboxy methylcellulose, polyethylene glycol, ethylcellulose, microcrystalline cellulose, polyvinyl pyrrolidone, polyvinyl alcohol, polyvinyl acetate, and ion exchange resins.

Carrier also includes all components of the coating composition, which may include plasticizers, pigments, colorants, stabilizing agents, and glidants. Foiniulations can be prepared using one or more pharmaceutically acceptable excipients, including diluents, preservatives, binders, lubricants, disintegrators, swelling agents, fillers, stabilizers, and combinations thereof.

Controlled release dosage formulations can be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on excipients, materials, equipment and process for preparing tablets and capsules and delayed release dosage forms of tablets, capsules, and granules. These references provide information on carriers, materials, equipment and process for preparing tablets and capsules and delayed release dosage forms of tablets, capsules, and granules.

Stabilizers are used to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

1. Brain Penetrating Formulations

In particularly preferred embodiments, the particles are formulated as brain penetrating particles. In specific embodiments, the brain penetrating particles have one, two, three, or all four of the following characteristics: (1) present a diameter less than about 100 nm when observed by transmission electron microscopy (TEM) or scanning electron microscopy (SEM), (2) have a hydrodynamic diameter less than about 200 nm when measured by dynamic light scattering (DLS), (3) have a neutral or negative surface charge, and (4) are non-aggregating after incubation in cerebrospinal fluid, artificial cerebrospinal fluid (aCSF) or other physiologically relevant serum at 37° C. for up to 24 hours.

2. Tropism of Target Cells

As illustrated in the Examples below, the surface chemistry of particles can influence their tropism for certain cells types in the brain, even absent specific or selective targeting moieties. For example, the experiments below illustrate that in healthy brain, PLA NPs appeared to be internalized homogeneously by a large number of cells, while fewer cells took up the PLA-HPG-CHO NPs but to a greater extent. Microglia activation was induced in PLA, PLA-HPG-CHO and PLA-PEG NP treated brains, while PLA-HPG NPs did not induce activation of microglia nor did they increase the presence of reactive astrocytes, even 24 h after introduction into the brain interstitium. Overall, these results show that in the healthy brain, untargeted PLA-PEG and PLA-HPG NPs were internalized substantially less compared to PLA NPs, and conversion of diols on HPG to aldehyde groups reversed and increased uptake in all cell types.

The experiments below also show that despite the varying amounts of uptake, all three tested particle types presenting surface modification (PLA-PEG, PLA-HPG and PLA-HPG-CHO NPs) displayed preferential uptake by tumor cells compared to other cell types after 24 h. The HPG surface modification was more efficient at decreasing microglia and neuron uptake compared to PEG, allowing for the highest specificity towards tumor cells, although the total uptake for both formulations (PLA-PEG and PLA-HPG NPs) was low. Substantial uptake by activated microglia and reactive astrocytes at the tumor periphery was observed, and the extent of uptake of all NPs types was substantially increased 24 h after introduction in the interstitial space, especially for tumor cells. Once again, the fraction associated with NPs depended on their surface properties.

Overall, normalization of total uptake for all particle types and conditions (healthy brain vs tumor-bearing brain, 4 h vs 24 h) showed that compared to PLA NPs, PLA-HPG-CHO displayed the highest internalization in all conditions, while PLA-PEG and PLA-HPG NPs presented the lowest uptake level. The higher internalization for PLA-HPG-CHO NPs extended to all cell types, including healthy cell populations (astrocytes, microglia and neurons).

Thus, depending on the target cell (e.g., tumor or healthy) and/or cell type (e.g., astrocytes, microglia and/or neurons) the surface chemistry of the particles can be taken into consideration in alternative or in addition to the selection of cell-specific or -selective targeting moieties, when preparing the particle formulation for a particular method of treatment.

### V. Method of Use

Methods of treating a subject in need by administering the subject an effective amount of the particles are provided. As generally used herein, an “effective amount” is that amount which is able to induce a desired result in a treated subject. The desired results will depend on the disease or condition to be treated. The condition or symptom can be a biochemical, molecular, physiological, or pathological readout. The precise dosage will vary according to a variety of factors such as subject-dependent variables (e.g., age, immune system health, etc.), the disease, and the treatment being effected. For example, therapeutically effective amounts of the disclosed particles used in the treatment of cancer will generally kill tumor cells or inhibit proliferation or metastasis of the tumor cells. Symptoms of cancer may be physical, such as tumor burden, or biological such as proliferation of cancer cells. The actual effective amounts of particles can vary according to factors including the specific particles administered, the particular composition formulated, the mode of administration, and the age, weight, condition of the subject being treated, as well as the route of administration and the disease or disorder. In exemplary embodiments, the particles are administered in an amount effective to kill cancer cells, improve survival of a subject with cancer, or a combination thereof. In a particular embodiment, the cancer is glioblastoma.

An effective amount of the particles can be compared to a control. Suitable controls are known in the art. A typical control can be a comparison of a condition or symptom of a subject prior to and after administration of the particles, or a comparison between one particle another. In the Examples below, particles modified to include a targeting moiety or a sensitizing agent are compared to unmodified particles. Particles with modified surface chemistries are compared to each other or unmodified particles. The particles can be otherwise the same, for example, composed of the same polymer, loaded with the same active agent, etc. In some embodiments, the effect of drug-loaded particles is compared to administration of free drug. In some embodiments, the control is the same particles administered by a different route or method of administration (e.g., CED vs. local injection). In another embodiment, the control is a matched subject that is administered a different therapeutic agent. Accordingly, the compositions disclosed here can be compared to other art recognized treatments for the disease or condition to be treated.

A. Subjects to be Treated

In general, the disclosed particles and methods of treatment thereof are useful in the context of cancer, including tumor therapy, particular brain tumor therapy. The particles can also be used for drug delivery for treatment of other diseases, disorders and injury including neurodegenerative diseases such as Parkinson's Alzheimer's, Huntington's, etc.; pediatric diseases and other lysosome storage diseases, including, but not limited to, Gaucher's disease, Hurler's disease, and Fabry's disease; genetic diseases; and cerebrovascular diseases and disorders.

1. Cancer

In a mature animal, a balance usually is maintained between cell renewal and cell death in most organs and tissues. The various types of mature cells in the body have a given life span; as these cells die, new cells are generated by the proliferation and differentiation of various types of stem cells. Under normal circumstances, the production of new cells is so regulated that the numbers of any particular type of cell remain constant. Occasionally, though, cells arise that are no longer responsive to normal growth-control mechanisms. These cells give rise to clones of cells that can expand to a considerable size, producing a tumor or neoplasm. A tumor that is not capable of indefinite growth and does not invade the healthy surrounding tissue extensively is benign. A tumor that continues to grow and becomes progressively invasive is malignant. The term cancer refers specifically to a malignant tumor. In addition to uncontrolled growth, malignant tumors exhibit metastasis. In this process, small clusters of cancerous cells dislodge from a tumor, invade the blood or lymphatic vessels, and are carried to other tissues, where they continue to proliferate. In this way a primary tumor at one site can give rise to a secondary tumor at another site.

The compositions and methods described herein are useful for treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth. The Examples below indicate that the particles and methods disclosed herein are useful for treating cancer, particular brain tumors, in vivo.

Malignant tumors that may be treated are classified herein according to the embryonic origin of the tissue from which the tumor is derived. Carcinomas are tumors arising from endodermal or ectodermal tissues such as skin or the epithelial lining of internal organs and glands. The disclosed compositions are particularly effective in treating carcinomas. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The types of cancer that can be treated with the provided compositions and methods include, but are not limited to, cancers, such as vascular cancer such as multiple myeloma; adenocarcinomas and sarcomas of bone, bladder, brain, breast, cervical, colo-rectal, esophageal, kidney, liver, lung, nasopharangeal, pancreatic, prostate, skin, stomach, and uterine. In some embodiments, the disclosed compositions are used to treat multiple cancer types concurrently. The compositions can also be used to treat metastases or tumors at multiple locations.

The disclosed methods are particularly useful in treating brain tumors. Brain tumors include all tumors inside the cranium or in the central spinal canal. They are created by an abnormal and uncontrolled cell division, normally either in the brain itself (neurons, glial cells (astrocytes, oligodendrocytes, ependymal cells, myelin-producing Schwann cells, lymphatic tissue, blood vessels), in the cranial nerves, in the brain envelopes (meninges), skull, pituitary and pineal gland, or spread from cancers primarily located in other organs (metastatic tumors). Examples of brain tumors include, but are not limited to, oligodendroglioma, meningioma, supratentorial ependymona, pineal region tumors, medulloblastoma, cerebellar astrocytoma, infratentorial ependymona, brainstem glioma, schwannomas, pituitary tumors, craniopharyngioma, optic glioma, and astrocytoma.

“Primary” brain tumors originate in the brain and “secondary” (metastatic) brain tumors originate from cancer cells that have migrated from other parts of the body. Primary brain cancer rarely spreads beyond the central nervous system, and death results from uncontrolled tumor growth within the limited space of the skull. Metastatic brain cancer indicates advanced disease and has a poor prognosis. Primary brain tumors can be cancerous or noncancerous. Both types take up space in the brain and may cause serious symptoms (e.g., vision or hearing loss) and complications (e.g., stroke). All cancerous brain tumors are life threatening (malignant) because they have an aggressive and invasive nature. A noncancerous primary brain tumor is life threatening when it compromises vital structures (e.g., an artery). In a particular embodiment, the disclosed compositions and methods are used to treat cancer cells or tumors that have metastasized from outside the brain (e.g., lung, breast, melanoma) and migrated into the brain.

The Examples below illustrate that the particles can be administered directly to the brain, for example, by CED. Therefore, the disclosed particles are particularly useful for treating brain cancer, and cancer that can metastasize to the brains, for example lung cancer, breast cancer, and skin cancer such as melanoma.

Although the particles are particularly safe and useful for treating cancer in the brain, the cancer does not have to be in the brain. Thus the particles can also be used for treating other cancer outside the brain, and can thereof be administered systemically in or locally outside the brain.

2. Neurodegenerative Diseases

The disclosed compositions and methods can also be used to delivery active agents for the treatment of a neurological or neurodegenerative disease or disorder or central nervous system disorder. Neurodegeneration refers to the progressive loss of structure or function of neurons, including death of neurons. For example, the compositions and methods disclosed herein can be used to treat subjects with a disease or disorder, such as Parkinson's Disease (PD) and PD-related disorders, Huntington's Disease (HD), Amyotrophic Lateral Sclerosis (ALS), Alzheimer's Disease (AD) and other dementias, Prion Diseases such as Creutzfeldt-Jakob Disease, Corticobasal Degeneration, Frontotemporal Dementia, HIV-Related Cognitive Impairment, Mild Cognitive Impairment, Motor Neuron Diseases (MND), Spinocerebellar Ataxia (SCA), Spinal Muscular Atrophy (SMA), Friedreich's Ataxia, Lewy Body Disease, Alpers' Disease, Batten Disease, Cerebro-Oculo-Facio-Skeletal Syndrome, Corticobasal Degeneration, Gerstmann-Straussler-Scheinker Disease, Kuru, Leigh's Disease, Monomelic Amyotrophy, Multiple System Atrophy, Multiple System Atrophy With Orthostatic Hypotension (Shy-Drager Syndrome), Multiple Sclerosis (MS), Neurodegeneration with Brain Iron Accumulation, Opsoclonus Myoclonus, Posterior Cortical Atrophy, Primary Progressive Aphasia, Progressive Supranuclear Palsy, Vascular Dementia, Progressive Multifocal Leukoencephalopathy, Dementia with Lewy Bodies (DLB), Lacunar syndromes, Hydrocephalus, Wernicke-Korsakoff s syndrome, post-encephalitic dementia, cancer and chemotherapy-associated cognitive impairment and dementia, and depression-induced dementia and pseudodementia.

In some embodiments, the subject has a central nervous system disorder or is in need of neuroprotection. Exemplary conditions and/or subjects include, but are not limited to, subjects having had, subjects with, or subjects likely to develop or suffer from a stroke, a traumatic brain injury, a spinal cord injury, Post-Traumatic Stress syndrome, or a combination thereof.

In some embodiments, the disclosed compositions and methods are administered to a subject in need thereof in an effective amount to reduce or prevent one or more molecular or clinical symptoms of a neurodegenerative disease, or one or more mechanisms that cause neurodegeneration. Neurodegeneration, and diseases and disorders thereof, can be caused by a genetic mutation or mutations; protein mis-folding; intracellular mechanisms such as dysregulated protein degradation pathways, membrane damage, mitochondrial dysfunction, or defects in axonal transport; defects in programmed cell death mechanisms including apoptosis, autophagy, cytoplasmic cell death; and combinations thereof. More specific mechanisms common to neurodegenerative disorders include, for example, oxidative stress, mitochondrial dysfunction, excitotoxicity, inflammatory changes, iron accumulation, and/or protein aggregation.

Symptoms of neurodegenerative diseases are known in the art and vary from disease to disease. In some embodiments, the disease exhibits or is characterized by one or any combination of the following symptoms or diseases: stress, anxiety, seasonal depression, insomnia and tiredness, schizophrenia, panic attacks, melancholy, dysfunction in the regulation of appetite, insomnia, psychotic problems, epilepsy, senile dementia, various disorders resulting from normal or pathological aging, migraine, memory loss, disorders of cerebral circulation, cardiovascular pathologies, pathologies of the digestive system, fatigue due to appetite disorders, obesity, pain, psychotic disorders, diabetes, senile dementia, or sexual dysfunction. In some embodiments, the subject does not exhibit one or more of the preceding symptoms.

In some embodiments, the subject has been medically diagnosed as having a neurodegenerative disease or a condition in need of neuroprotection by exhibiting clinical (e.g., physical) symptoms of the disease. Therefore, in some embodiments, the compounds or compositions disclosed herein are administered prior to a clinical diagnosis of a disease or condition. In some embodiments, a genetic test indicates that the subject has one or more genetic mutations associated with a neurodegenerative disease or central nervous system disorder.

Neurodegenerative diseases are typically more common in aged individuals. Therefore in some embodiments, the subject is greater the 40, 45, 50, 55, 60, 65, 70, 75, 80, 85, 90, 95, 100 years in age.

Active agents for the treatment of neurodegenerative diseases are well known in the art and can vary based on the symptoms and disease to be treated. For example, conventional treatment for Parkinson's disease can include levodopa (usually combined with a dopa decarboxylase inhibitor or COMT inhibitor), a dopamine agonist, or an MAO-B inhibitor.

Treatment for Huntington's disease can include a dopamine blocker to help reduce abnormal behaviors and movements, or a drug such as amantadine and tetrabenazine to control movement, etc. Other drugs that help to reduce chorea include neuroleptics and benzodiazepines. Compounds such as amantadine or remacemide have shown preliminary positive results. Hypokinesia and rigidity, especially in juvenile cases, can be treated with antiparkinsonian drugs, and myoclonic hyperkinesia can be treated with valproic acid. Psychiatric symptoms can be treated with medications similar to those used in the general population. Selective serotonin reuptake inhibitors and mirtazapine have been recommended for depression, while atypical antipsychotic drugs are recommended for psychosis and behavioral problems.

Riluzole (RILUTEK®) (2-amino-6-(trifluoromethoxy) benzothiazole), an antiexcitotoxin, has yielded improved survival time in subjects with ALS. Other medications, most used off-label, and interventions can reduce symptoms due to ALS. Some treatments improve quality of life and a few appear to extend life. Common ALS-related therapies are reviewed in Gordon, Aging and Disease, 4(5):295-310 (2013), see, e.g., Table 1 therein. A number of other agents have been tested in one or more clinical trials with efficacies ranging from non-efficacious to promising. Exemplary agents are reviewed in Carlesi, et al., Archives Italiennes de Biologie, 149:151-167 (2011). For example, therapies may include an agent that reduces excitotoxicity such as talampanel (8-methyl-7H-1,3-dioxolo(2,3)benzodiazepine), a cephalosporin such as ceftriaxone, or memantine; an agent that reduces oxidative stress such as coenzyme Q10, manganoporphyrins, KNS-760704 [(6R)-4,5,6,7-tetrahydro-N6-propyl-2,6-benzothiazole-diamine dihydrochloride, RPPX], or edaravone (3-methyl-1-phenyl-2-pyrazolin-5-one, MCI-186); an agent that reduces apoptosis such as histone deacetylase (HDAC) inhibitors including valproic acid, TCH346 (Dibenzo(b,f)oxepin-10-ylmethyl-methylprop-2-ynylamine), minocycline, or tauroursodeoxycholic Acid (TUDCA); an agent that reduces neuroinflammation such as thalidomide and celastol; a neurotropic agent such as insulin-like growth factor 1 (IGF-1) or vascular endothelial growth factor (VEGF); a heat shock protein inducer such as arimoclomol; or an autophagy inducer such as rapamycin or lithium.

Treatment for Alzheimer's Disease can include, for example, an acetylcholinesterase inhibitor such as tacrine, rivastigmine, galantamine or donepezil; an NMDA receptor antagonist such as memantine; or an antipsychotic drug.

Treatment for Dementia with Lewy Bodies can include, for example, acetylcholinesterase inhibitors such as tacrine, rivastigmine, galantamine or donepezil; the N-methyl d-aspartate receptor antagonist memantine; dopaminergic therapy, for example, levodopa or selegiline; antipsychotics such as olanzapine or clozapine; REM disorder therapies such as clonazepam, melatonin, or quetiapine; anti-depression and antianxiety therapies such as selective serotonin reuptake inhibitors (citalopram, escitalopram, sertraline, paroxetine, etc.) or serotonin and noradrenaline reuptake inhibitors (venlafaxine, mirtazapine, and bupropion) (see, e.g., Macijauskiene, et al., Medicina (Kaunas), 48(1):1-8 (2012)).

Exemplary neuroprotective agents are also known in the art in include, for example, glutamate antagonists, antioxidants, and NMDA receptor stimulants. Other neuroprotective agents and treatments include caspase inhibitors, trophic factors, anti-protein aggregation agents, therapeutic hypothermia, and erythropoietin.

Other common active agents for treating neurological dysfunction include amantadine and anticholinergics for treating motor symptoms, clozapine for treating psychosis, cholinesterase inhibitors for treating dementia, and modafinil for treating daytime sleepiness.

3. Cerebrovascular Diseases and Disorders

The compositions and methods can also be used to treat cerebrovascular diseases and symptoms and complications thereof including stroke, cerebrovascular accident, hypertension, artherosclerosis, high cholesterol, inflammation, dementia, cerebral thrombosis, cerebral embolism, cerebral hemorrhage, aneurysms. General signs and symptoms of a hemorrhagic or ischemic event include motor dysfunction, such as hemiplegia and hemiparesis. Active agents for treatment can include, for example, antiplatelets (Aspirin, Clopidogrel), blood thinners (Heparin, Warfarin), antihypertensives (ACE inhibitors, Beta blockers), an anti-diabetic medications.

B. Methods of Treatment

The particles are preferably administered into or adjacent to the area to be treated, for example, the area of the CNS (or the brain) to be treated. This may be at the time of or immediately after surgical resection of a tumor. Preferably, the particles are administered by injection into the tissue or the blood vessels leading into the brain. Particles can be introduced directly in the tissue by direct infusion or convection-enhanced delivery (CED). Alternately, they can be administered intravenously, or intra-arterially via catheter into an artery that serves the region of the tissue to be treated.

To overcome the challenges associated with drug delivery to the brain or other regions of the central nervous system, a controlled-release delivery system is provided that includes brain-penetrating polymeric nanoparticles. Small particles (e.g., less than about 100 nm) and an aggregation reducing agent each alone, and even more substantially when used in combination, can increase the penetration of particles into the brain, particularly when delivered intracranially using CED (U.S. Published Application No. 2015-0118311). The penetration of these particles is as good as any previously reported nanoparticle systems: for example, the V_{d}/Vᵢ achieved are comparable to those achieved with nanoliposomal delivery systems in rats. The Examples below show that including a targeting moiety or sensitizing agent can dramatically increase the tumor targeting and treatment efficacy of chemotherapeutic drugs.

Polymeric particles have many advantages over liposomal formulations including lower toxicity and control of drug release. PLGA nanoparticles delivered in pig brains using CED penetrated to volumes of approximately 1180 mm³. Since the vast majority of GBMs recur within 2 cm of the original tumor focus, the penetrative capacity of these brain-penetrating nanoparticles when delivered by CED can address the infiltrative nature of GBM. Surface-modified nanoparticles with an imaging agent, for example [¹⁸F]NPB4 using streptavidin-biotin conjugation, allows tracking the nanoparticles during the CED procedure using non-invasive PET imaging. This allows clinicians to visualize nanoparticles delivered by CED and ensure distribution of the therapeutic agent throughout the brain regions most likely in need of treatment.

### EXAMPLES

### Example 1: Hyperbranched Polyglycerol (HPG) Coating Alters Cellular Tropism of Poly(Lactic Acid) (PLA) Nanoparticles

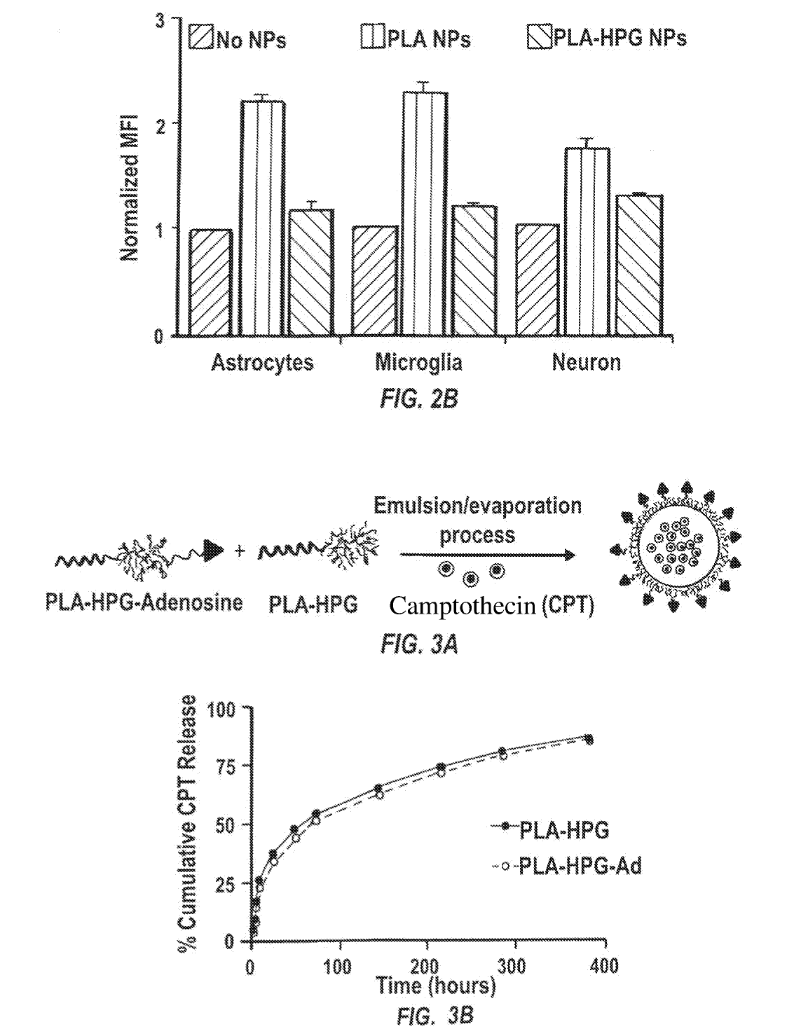

Poly(lactic acid)-hyperbranched polyglycerol (PLA-HPG) nanoparticles were prepared by an emulsion/evaporation process to yield particles in the size range of 100 nm to 150 nm and loaded with fluorescent dye. FIG. 2A shows that HPG coating does not modify the volume of distribution of the nanoparticles. FIG. 2B shows that HPG coating influences cellular tropism relative to at least astrocytes, microglia, and neurons.

### Example 2: Functionalization of PLA-HPG with Adenosine Increases Survival in a Rat Model of Glioma

Brain diseases represent a major health concern, due to their gravity and the socio-economical burden that they imply. In particular, it is estimated that 70,000 people worldwide will be diagnosed with a primary cerebral tumor each year. Amongst these patients, 17% are suffering from glioblastoma multiform (GBM), the most aggressive grade of glioma (Ostrom Q T et al., Cancer Treat Res, 163: 1-14 (2015)). Despite major progress in the development of new chemotherapeutic drugs and improved surgical technics, GBM prognostic remains poor, with a median survival of 15 months (Wen P Y et al., N Engl J Med, 359: 492-507 (2008)). This is due to two major hurdles in brain tumor patients' care. First, drug delivery remains one of the main challenges of central nervous system (CNS) drug development, because of the fast metabolism and/or rapid clearance of most CNS drugs, as well as their generally poor permeation through the blood-brain barrier (BBB) (Pardridge W M, J Cereb Blood Flow Metab, 32: 959-972 (2012)). Another difficulty in the case of brain cancer is the systemic toxicity of chemotherapeutic molecules, limiting the amount of drug that can be administered safely to the patient. Second, despite a standard therapy involving surgical resection when feasible, radiotherapy and chemotherapy, 90% of the tumors recur within 2 cm of their original site, accounting for the very short survival (Wen P Y et al., N Engl J Med, 359: 492-507 (2008)). Cancer stem cells (CSC) are postulated mediators of chemoresistance and relapse (Reya T et al., Nature 414: 105-111 (2001); Beier D et al., Mol Cancer 10: 128 (2011)). Their ability to resist chemotherapeutic treatments and radiation might be due to their enhanced capacity to expel cytotoxic drugs (Hirschmann-Jax C et al., Proc Natl Acad Sci USA 101: 14228-14233 (2004)), their increased DNA repair capacity (Bao S et al., Nature 444: 756-760 (2006)) and their low mitotic activity. Another important feature of CSCs is the degree to which they are regulated by their immediate environment (Calabrese C et al., Cancer Cell 11: 69-82 (2007)). In particular, the brain tumor stroma, including astrocytes, endothelial cells, pericytes and microglia, is able to enrich the microenvironment with soluble factors, forming a tumoral niche that promotes CSC survival and proliferation (Jones T S and Holland E C, Oncogene 31: 1995-2006 (2012)). CSCs are postulated mediators of chemoresistance in brain tumors and relapse (Reya T et al., Nature 414: 105-111 (2001)). It has been proposed that two strategies could be used to kill CSCs and prevent recurrence (Dirks P B., Mol Oncol 4: 420-430 (2010)): (1) the promotion of CSC differentiation to make them sensitive to chemotherapeutic drugs, and (2) blocking interactions between CSCs and the tumoral niche. Adenosine has recently been shown to address these two strategies thanks to its pleiotropic and multitargeted activity. First, the activation of adenosine receptors A₁ and A_{2B} was able to induce CSC differentiation and to sensitize CSCs to the chemotherapeutic activity of temozolomide (Daniele S et al., Cell Death Dis 5: e1539 (2014)). Second, it has been shown that the activation of the A₁ receptor plays an antitumorigenic role, which is mediated by microglia cells (Synowitz M et al., Cancer Res 66: 8550-8557 (2006)). Altogether, these results strongly indicate that modulating the activity of the adenosine receptors may be an efficient strategy to sensitize CSC to chemotherapy.

### Materials and Methods

Functionalized PLA-HPG Synthesis

The functionalization of PLA-HPG polymers was obtained by coupling the hydroxyl groups of the polyglycerol moieties of the HPG with a carboxylic group on the ligand. PLA-HPG polymer was functionalized with the small molecule adenosine, under a carboxylic modified form (2′,3′-isopropylidene adenosine-5′-carboxylic acid), using the following procedure: 500 mg PLA-HPG is added to 40 mg 2′,3′-isopropylidene adenosine-5′-carboxylic acid and dissolved in anhydrous DMF. The solution is dried with a molecular sieve with 39 μL DIC and 6 mg DMAP added to the solution. To purify the polymer, the solution is added into cold diethyl ether to precipitate the polymer. The polymer precipitate is collected and dissolved in 3 mL DCM/TFA mixture (DCM:TFA=2:1) and the reaction shaken at room temperature for 2 h. The resulting solution is added into cold diethyl ether and the polymer collected by centrifugation. The polymer is further purified by redissolving in DCM and precipitating in diethyl ether. To confirm conjugation of Ad to PLA-HPG, the polymers is dissolved in DMSO-d6 and ¹H NMR analysis is performed. The PLA-HPG-Adenosine polymer is then used to form PLA-HPG-Adenosine NPs using the emulsion solvent evaporation technique.

PLA-HPG-Ad Nanoparticles Preparation

PLA-HPG-Ad NPs were synthesized using the emulsion-evaporation technique, as previously described (Deng, et al., Biomaterials, 35:6595-6602 (2014)). 90 mg of PLA-HPG and 10 mg of PLA-HPG-Ad were dissolved in 2.4 mL of ethyl acetate overnight. 10 mg of camptothecin (CPT) (Sigma-Aldrich, C9911) was dissolved in 600 μL DMSO and added to the polymer solution. The organic phase was then added dropwise to 4 mL of DI water under a strong vortex, and the mixture was sonicated for four cycles of 10 sec intervals before dilution in another 10 mL of DI water. The final mixture was concentrated using a rotovap for 15 min at RT. Following evaporation, the solution was transferred to an Amicon Ultra-15 100 kDa centrifugal filter unit, and centrifuged at 3000 g at 4° C. for 45 min. The NPs were washed twice with 15 mL DI water and centrifuged for 45 min each time. Subsequently, the NPs were resuspended in 1 mL DI water, and snap-frozen in aliquots until use.

Drug Release Study

Release from PLA-HPG and PLA-HPG-Ad NPs was performed under in vitro physiological conditions with triplicate samples. NPs suspended in water were placed in dialysis tubing (10K cut-off) under sink conditions in 40 ml of sterile PBS at 37° C. with stirring. At designated timepoints, the dialyzate was reserved for analysis and replaced with 40 ml of fresh PBS. To quantify the amount of CPT released from the NPs, 970 μl of dilyzate from each sample and timepoint was added to 30 μl of acidified buffer (DMSO:10% SDS:1N HCl at a 1:1:1 volume ratio). The CPT concentration was detected using a Molecular Devices SpectraMax M5 plate reader at ex/em 370/428 and compared to NP loading to quantify total release.

Orthotopic Tumor Inoculation

Orthotopic RG2 tumors were inoculated in Fischer 344 rats as previously described (Saucier-Sawyer et al., J Controlled Release, 232: 103-112 (2016)). Animals were anesthetized using a mixture of ketamine (75 mg/kg) and xylazine (5 mg/kg), injected intraperitoneally. Rats' heads were shaved and then placed in a stereotaxic frame. After sterilization of the scalp with alcohol and betadine, a midline scalp incision was made to expose the coronal and sagittal sutures, and a burr hole was drilled 3 mm lateral to the sagittal suture and 0.5 mm anterior to the bregma. A 10 μL Hamilton syringe, loaded with RG2 cells, was inserted into the burr hole at a depth of 5 mm from the surface of the brain and left to equilibrate for 5 min before infusion. A micro-infusion pump (World Precision Instruments, Sarasota, Fla., USA) was used to infuse 3 μL of cell suspension (250,000 cells total) at a rate of 1000 μL/min. Once the infusion was finished, the syringe was left in place for another 5 min before removal of the syringe. Bone wax was used to fill the burr hole and skin was stapled and cleaned. Following intramuscular administration of analgesic (Meloxicam, 1 mg/kg), animals were placed in a heated cage until full. Tumors were grown for 4 days before administration of particles.

Convection enhanced delivery of particles in the tumor bearing brain CED in tumor bearing rats was conducted following a similar procedure as for tumor implantation, by reopening the burr hole used for tumor implantation. A 50 μL Hamilton syringe with a polyamide-tipped tubing, loaded with the PBS, PLA-HPG CPT loaded NPs or PLA-HPG-Ad CPT loaded NPs, was inserted into the hole at a depth of 5 mm from the surface of the brain and left to equilibrate for 7 min before infusion. A micro-infusion pump (World Precision Instruments, Sarasota, Fla., USA) was used to infuse 20 μL of 50 mg/mL NPs at a rate of 0.667 μL/min. Once the infusion was finished, the syringe was left in place for another 7 min before removal of the syringe.

### Results

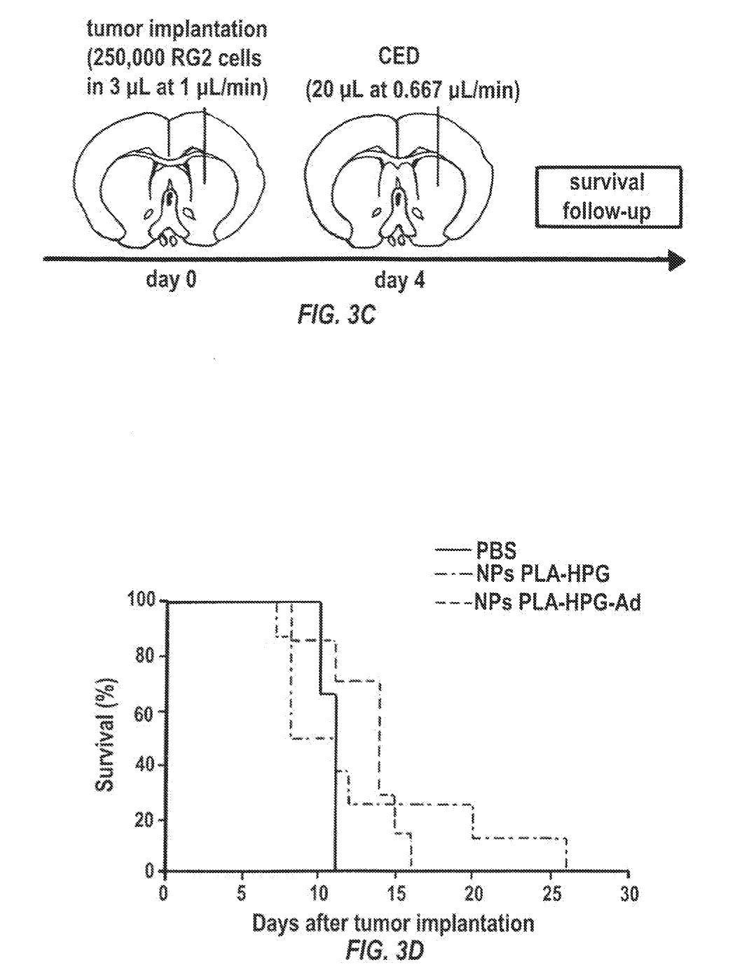

FIG. 3B shows that the addition of adenosine does not substantially affect the release of camptothecin (CPT) from PLA-HPG nanoparticles. Table 1 shows that the addition of adenosine does not substantially affect the diameter or of PLA-HPG CPT loaded nanoparticles.

TABLE 1
Nanoparticle characteristics.

| | PLA-HPG NPs | PLA-HPG-Ad NPs |
|---|---|---|
| Diameter (nm) | 54.3 | 55.4 |
| CPT loading | 16% | 11% |

FIG. 3D shows that CPT-loaded PLA-HPG particles extend survival compared to PBS treated controls, and CPT-loaded PLA-HPG-Ad particles further extend survival compared to CPT-loaded PLA-HPG particles, when the agents are delivered to the brain by convection-enhanced delivery (CED) in a rat model of GBM (FIG. 3C).

### Example 3: Functionalization with pHILP Induces Particle Accumulation at the Periphery of the Tumor

One of the hallmarks of solid tumor microenvironment is acidosis, which is mainly caused by lactic acid accumulation in rapidly growing tumor cells, together with insufficient blood supply and poor lymphatic drainage (Kim J W et al., Cancer Res 66: 8927-8930 (2006); Brahimi-Hom M C et al., FEBS Lett 581: 3582-3591(2007)). Recent studies showed that gliomas, like other solid tumors, have acidic microenvironments (Gerweck L E et al., Cancer Res 56: 1194-1198 (1996)). pHLIP (pH Low Insertion Peptides) is a 36-amino acid peptide that adopts an α-helical conformational change at low pH (<6.5), facilitating insertion of its C-terminus into lipid bilayers (An M et al., Proc Natl Acad Sci USA 107: 20246-20250 (2010)). pHLIP has been shown to target multiple tumor types when administered systemically (Andreev O A et al., Proc Natl Acad Sci USA 104: 7893-7898 (2007)). In the case of brain tumors, pHLIP can target NPs to the tumoral environment.

### Materials and Methods

pHLIP Peptide

The pHLIP peptide used in these experiments is AAEQNPIYWARYADWLFTTPLLLLDLALLVDADEGTG (SEQ ID NO:1)

Functionalized Nanoparticle Preparation

PLA-HPG NPs were prepared by the emulsion-evaporation technique described above. Paclitaxel (PTX) or DiD dye were loaded into the nanoparticles using the same method as CPT encapsulation. Functionalization of pre-formed PLA-HPG NPs were realized using a Schiff base reaction: the hydroxyl groups of the polyglycerol moieties of the HPG at the surface of the NPs are first turned into aldehyde groups and further react with an amine group on the ligand. PLA-HPG NPs are functionalized with a pH-sensitive peptide, pHLIP: the PLA-HPG NPs are prepared using the emulsion solvent evaporation technique. They are then rendered “sticky” by converting the alcohol groups of the HPG into aldehydes using NaIO₄. Following this treatment, the NaIO₄ is quenched using Na₂SO₃ and the NPs are incubated with pHLIP to induce a Schiff base reaction between the amino-oxy group on the pHLIP N-terminus and the aldehyde groups. After incubation, the unreacted pHLIP is washed by centrifugation, and the remaining aldehyde groups on the HPG are reconverted to alcohol using H₃NO.

In Vitro Cytotoxicity of PTX Loaded Particles

RG2 glioma cells were plated at a density of 5000 cells/well in 96-well plates. 24 h after, cells were treated with free PTX, PLA-HPG NPs or PLA-HPG-pHLIP NPs at PTX concentrations ranging from 0-100 uM. 48 h after incubation, cell viability was measured with CellTiter-Glo Luminescent Cell Viability Assay.

In Vitro Uptake of PLA-HPG-pHLIP Particles

RG2 cells were plated at a density of 100,000 cells/well in 12-well plates. 24 h after, cells were incubated with DiD-loaded PLA-HPG with or without pHLIP in either pH 7.4 or 6.3 culture media. 2 h after incubation, cells were washed 3 times with PBS and harvested for flow cytometry. Mean fluorescence intensities were determined by flow cytometry performed using an Attune NxT (Invitrogen, Carlsbad, Calif.).

In Vivo Uptake and Distribution of PLA-HPG-pHLIP Particles in the Tumor Bearing Brain

Innoculation of RG2-GFP cells and CED of PLA-HPG NPs or PLA-HPG-pHLIP NPs were after 7 days of tumor growth were performed as described above. 2 h after the end of CED, animals were euthanized and brains harvested for flow cytometry. Brains were stored in HBSS buffer on ice before preparation. The contralateral hemisphere and cerebellum were removed and 3 mm around the periphery of the injection site was obtained. Tissue was first incubated in 1 mg/mL DNAse and collagenase type II in 37° C. for 30 minutes. Tissue was cut into smaller pieces and serially pipetted using a 25 mL, 5 mL and 1 mL pipette with excess volume of HBSS. The single cell suspension was then put through a 40 μm cell strainer. Cells were collected by centrifugation under 1000 g for 10 minutes. Cells were resuspended in ice cold 1% BSA solution and viability was checked with a trypan blue staining under a hemocytometer. Cells were diluted to roughly 200,000 cells per mL and used Attun NxT for analysis. For evaluation of NP distribution in the tumor, brains were harvested immediately after infusion and flash frozen, before being sliced in 100 μl m slices and imaged.

### Results

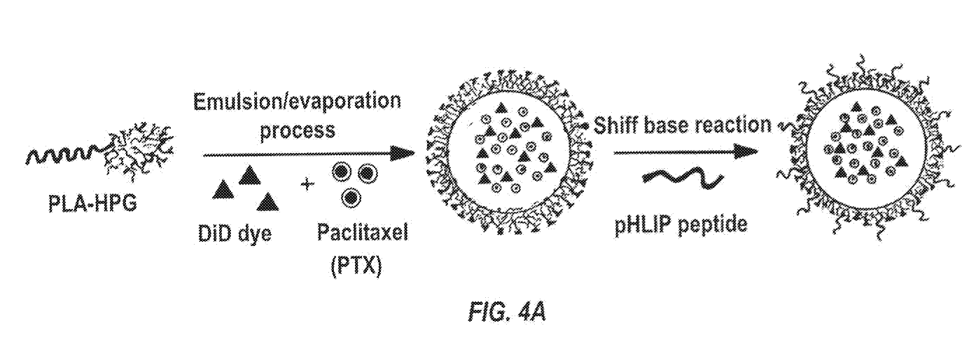

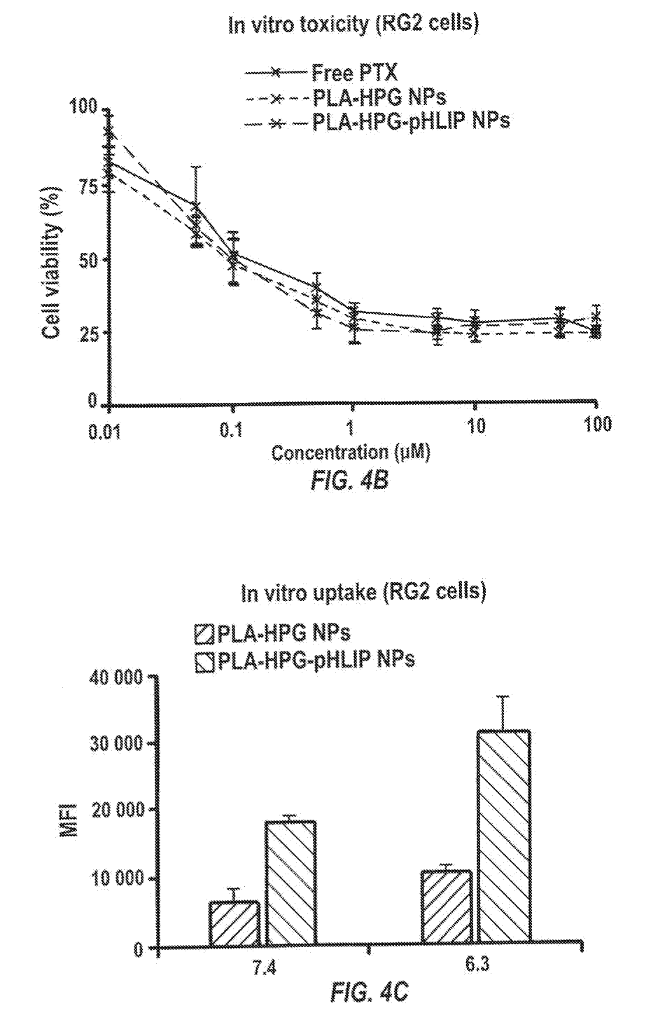

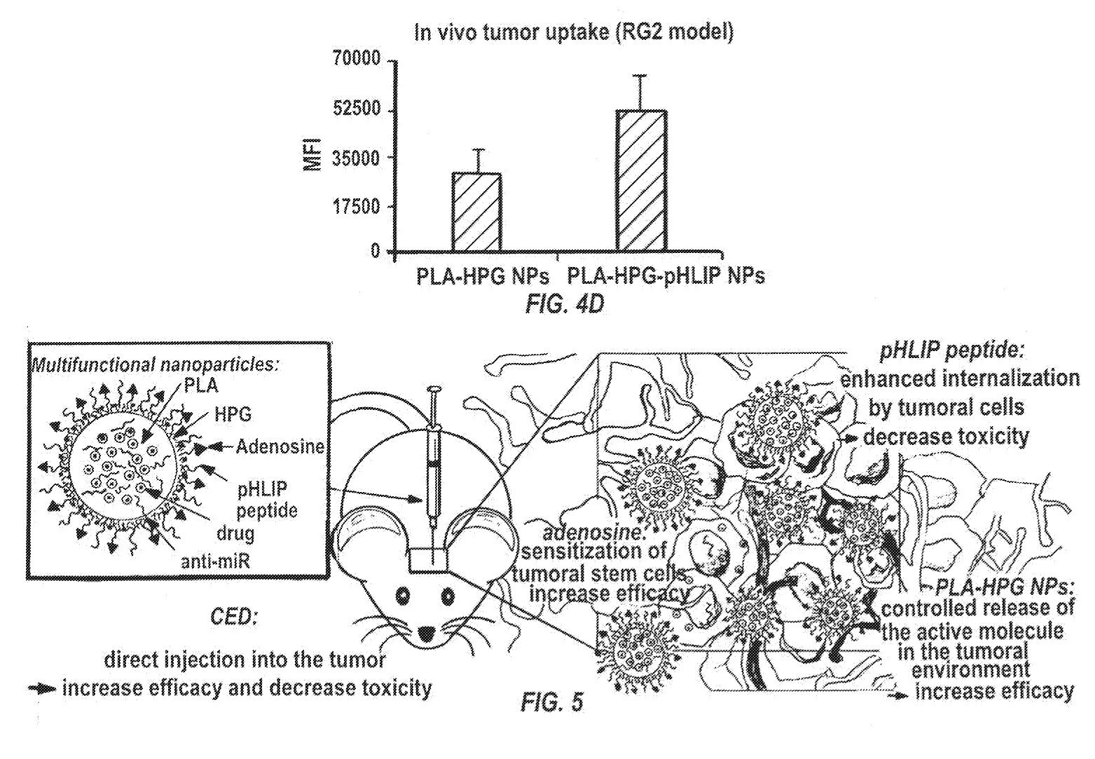

FIG. 4B shows that the addition of pHLIP does not influence activity of an encapsulated chemotherapeutic drug. Conjugation of pHLIP to the surface of NPs enhances internalization of NPs in tumor cells, and this increase in uptake is amplified in acidic pH (e.g., pH 6.3) in vitro (FIG. 4C). When delivered to animals with GFP-labeled tumors, the addition of pHLIP leads to a significant increase in uptake (FIG. 4D). Histological analysis of the tumors revealed that the addition of pHLIP induces particle accumulation at the periphery of the tumor.

An illustration of an exemplary multifunctional poly(lactic acid)-hyperbranched polyglycerol (PLA-HPG) nanoparticle, decorated with adenosine and pHILP peptide, and loaded with a small drug and an anti-miR nucleic acid is shown in FIG. 5. Increased antitumor efficacy can be achieved through controlled release of the particle contents through use of PLA-HPG polymers, sensitization of tumoral stem cells by functionalizing the particles with adenosine, and increased internalization in tumor cells by functionalizing the particles with pHILP, particularly when the particles are delivered locally to a brain tumor using convection-enhanced delivery (CED).

### Example 4: Characterization of Brain Penetrating Nanoparticles Bearing Different Surface Coatings

Recent developments in personalized medicine and drug delivery have produced exciting opportunities in oncology (Ferrari, et al., Nat Rev Cancer, 5:161-171 (2005)). Despite these advances, as well as major progress in the development of new chemotherapeutics and improved surgical techniques, prognosis for individuals with high-grade glioma, such as glioblastoma multiforme (GBM), remains poor, with a median survival of 15 months (Wen, et al., N Engl J Med, 359:492-507 (2008)). Among the barriers preventing effective treatment of brain tumors, the blood brain barrier (BBB) is the most prominent (Pardridge, et al., NeuroRx, 2:3-14 (2005)), hampering the achievement of relevant therapeutic concentrations of drug in the tumor mass without inducing systemic cytotoxicity. More generally, the BBB selectivity inhibits therapy efficacy in brain tumors, neurodegenerative diseases, stroke, and every major condition that afflicts the central nervous system (CNS).

Nanomaterials have long been proposed as carriers to facilitate the entry and delivery of agents into the brain (Cheng, et al., Nat Rev Drug Discov, 14:239-247 (2015); Gao, et al., Pharm Res, 30:2485-2498 (2013); Kreuter, et al., Adv Drug Deliv Rev, 71:2-14 (2014)). Long circulating nanoparticles (NPs), such as those decorated with polyethylene glycol (PEG) on their surface, have been proposed as a way to enhance brain penetration through the BBB.

However, despite their so-called “stealth” properties (due to their resistance to opsonization and further elimination by the reticuloendothelial system (Gref, et al., Adv Drug Deliv Rev, 16:215-233 (1995)), usually less than 1% of the injected dose gains access to the brain parenchyma after systemic administration (Gao, et al., Pharm Res, 30:2485-2498 (2013); Kreuter, et al., Advanced Drug Delivery Reviews, 71:2-14 (2014)). Transport through the nasal epithelium can be more efficient, although the small surface area and anatomical location tend to limit the value of this method (Pardridge, et al., Drug Discov Today, 12:54-61 (2007)). Local infusion directly into the brain, or convection-enhanced delivery (CED) (Bobo, et al., Proc. Nat. Acad. Sci., 91:2076-2080 (1994)), can be used to slowly introduce large volumes of NPs into the cerebral interstitium, allowing the NPs to reside in the brain parenchyma while slowly releasing encapsulated agents over prolonged periods (Sawyer, et al., Drug Delivery and Translational Research, 1:34-42 (2011); Zhou, et al., Proceedings of the National Academy of Sciences, 110:11751-11756 (2013)). It has been proposed that in order to be efficient when administered by CED, NPs need to penetrate readily through the brain tissue, or be “brain-penetrating” (Zhou, et al., Proceedings of the National Academy of Sciences, 110:11751-11756 (2013); Phillips, et al., Neuro Oncol, 14:416-425 (2012); Nance, et al., Sci Transl Med, 4 (2012)). Dense PEG surface coating of NPs has been used to increase the distribution of particles in the brain after CED (15), extending stealth properties to the brain interstitial space by preventing adhesive trapping in the extracellular matrix.

### Materials and Method

NPs Preparation

PLA NPs and PLA-HPG NPs

PLA and PLA-HPG NPs were synthesized using the emulsion-evaporation technique, as described above

PLA-PEG NPs PLA-PEG NPs were synthesized using a nanoprecipitation technique. 100 mg of polymer was dissolved in 5 mL DMSO at RT for 2 h. 0.2 mg of DiA dye dissolved in 2 μL of DMSO was then added to the polymer solution. The polymer-dye solution was then divided into 200 μL it aliquots. Each 200 μL aliquot was added drop-wise to 1 mL DI water under strong vortex to create a NP suspension. These suspensions were immediately pooled and diluted with 5× DI water. This diluted suspension was then transferred to an Amicon Ultra-15 100 kDa centrifugal filter unit, and centrifuged at 4000 g at 4° C. for 30 min. The NPs were washed twice with DI water and centrifuged for another 30 min each time. After a final wash with DI water, the NPs were centrifuged for 1 h to achieve a final concentration of 100 mg/mL DI water. The final NP suspension was then either immediately used for in vivo or in vitro experiments, or snap-frozen at −80° C. until use.

PLA-HPG-CHO NPs

PLA-HPG-CHO NPs were prepared by oxidation of the vicinal diols of PLA-HPG NPs to aldehydes as previously described with minor modifications24. 200 μL of PLA-HPG NPs at 100 mg/mL were incubated for 20 min on ice with 60 μL of 10×PBS and 200 μL of 0.1M NaIO4(aq). The reaction was then quenched with 200 μL of 0.2M Na2SO3(aq) and the NPs were washed two times with DI water using Amicon Ultracel 100 kDa MWCO centrifugal filter units at 12,200 g for 7 min each. The NPs were then diluted with DI water to desired concentrations for experiments.

NPs Characterization

Size and Zeta Potential Measurements

The hydrodynamic diameter of the NPs was measured by Dynamic Light Scattering (DLS). 1 mL of NPs (0.05 mg/mL in DI water) was prepared and read on a Malvern Nano-ZS (Malvern Instruments, UK). To measure zeta potential, 750 μL of NPs (0.05 mg/mL in DI water) were loaded into a disposable capillary cell and analyzed on a Malvern Nano-ZS.

TEM Imaging

For TEM imaging, 10 μL of NP solution at a concentration of 10 mg/mL was placed on a pre-cleaned and hydrophilized CF400-CU TEM grid (Electron Microscopy Sciences, Hatfield, Pa.) for 1 min. Grids were stained with a 0.2% uranyl acetate solution for 15 s, washed three times in DI water, and mounted for imaging with a Tecnai T12 TEM microscope (FEI, Hillsboro, Oreg.).

Particle Stability in aCSF

Particles were measured using Malvern Nano-ZS in artificial cerebrospinal fluid (aCSF; Harvard Apparatus, Holliston, Mass.) at 37° C. with a standard operating procedure taking measurements every minute.

Particle Loading and Yield

A 100 μL solution of NPs was lyophilized in a pre-weighed eppendorf tube to measure particle yield. Dye loading was determined using a SpectraMax M5 plate reader (Molecular Devices, Sunnyvale, Calif.) at 456/590 (nm).

Volume of Distribution

CED of NPs was performed in healthy Fischer 344 rats as described above. Brains were harvested immediately after infusion and flash frozen, before being sliced in 50 μm slices using a Leica Cryostat CM3000 (Leica, Germany). Slides were imaged using a Zeiss Lumar.V12 stereoscope (Carl Zeiss AG, Germany) and images were analyzed using a MATLAB code setting a threshold with Otsu's method.

### Results

Composed of one of the few degradable polymers approved by the FDA for medical applications (Marin, et al., Int J Nanomedicine, 8:3071-3090 (2013)) PLA NPs have been extensively studied because of their biodegradability and versatility, including for the treatment of neurological diseases.

Four PLA-based NP formulations were compared. PLA-PEG NPs are in advanced clinical trials for systemic delivery of chemotherapeutics (Harris, et al., Nat Rev Drug Discov, 2:214-221 (2003)), however, PEG itself presents the disadvantage of a non-biodegradable main chain, and the possible induction of an anti-PEG immune response (Garay, et al., Expert Opin Drug Deliv, 9:1319-1323 (2012)). As an alternate, hyperbranched glycerol (HPG) can also be used as a surface coating of PLA NPs: PLA-HPG NPs (Deng, et al., Biomaterials, 35:6595-6602 (2014)) produce a greater stealth effect than PLA-PEG, avoid recognition by phagocytic cells, and provide multiple functionalizing sites. PLA-HPG NPs have the additional advantage that they can be turned into bioadhesive NPs (Deng, et al., Nat Mater, 14:1278-1285 (2015)), by converting the vicinal diols of the HPG into aldehydes (—CHO), obtaining PLA-HPG-CHO NPs.

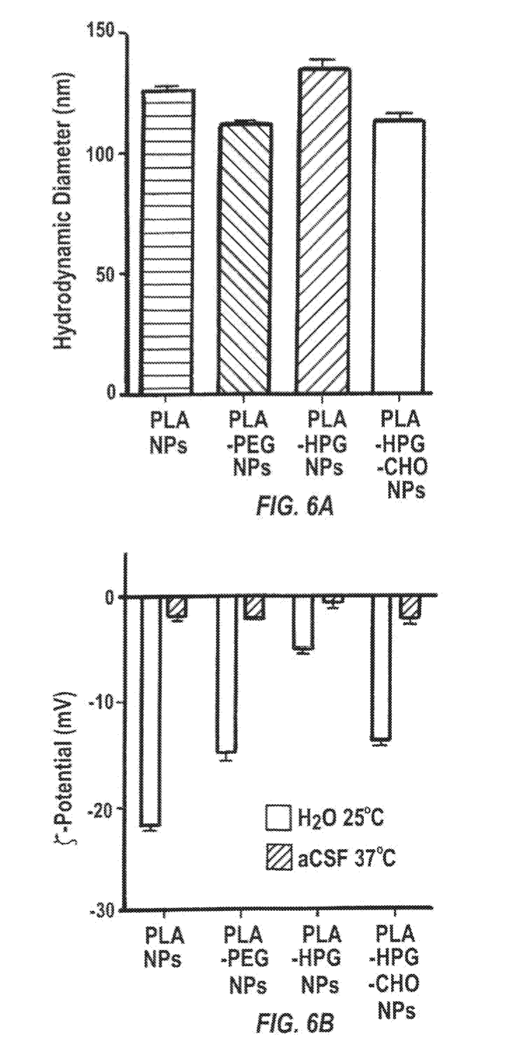

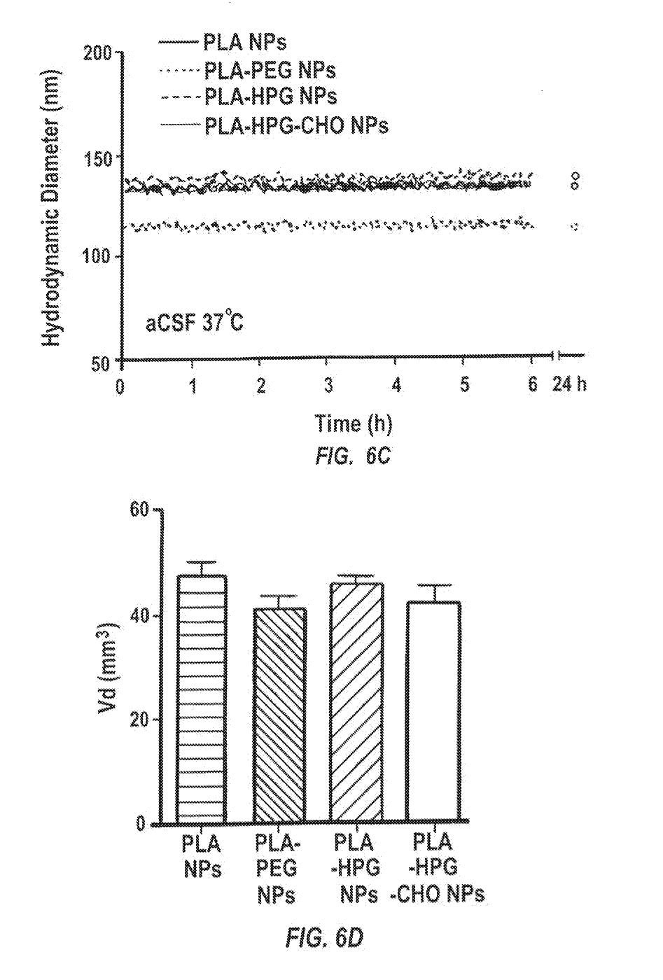

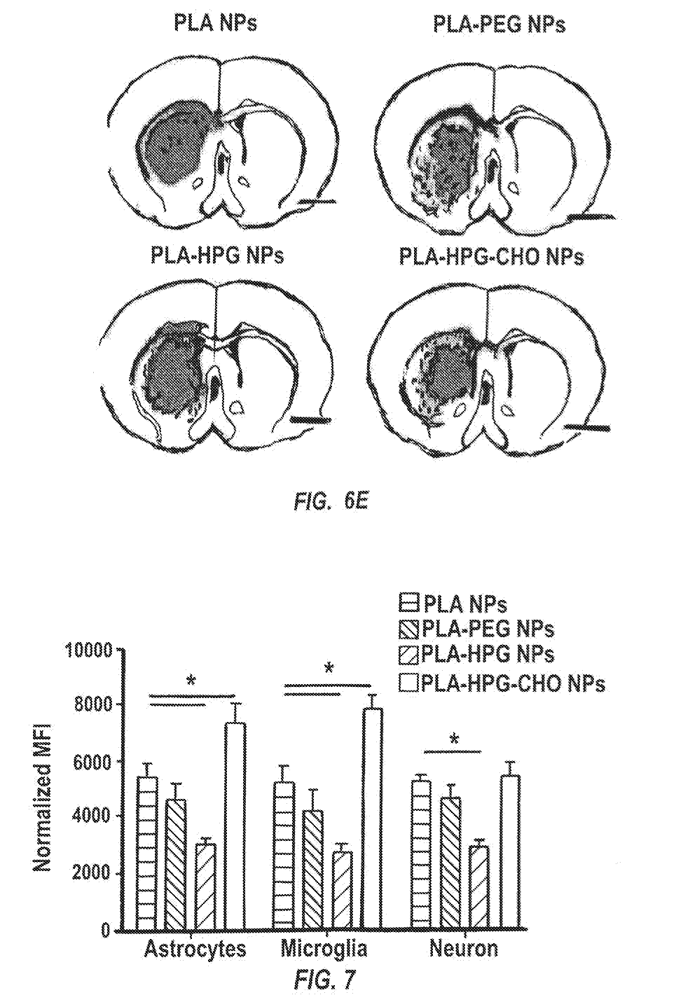

Each of these four NP formulations (PLA, PLA-PEG, PLA-HPG, and PLA-HPG-CHO NPs) were successfully engineered to fit the criteria to be brain penetrating (Zhou, et al., Proceedings of the National Academy of Sciences, 110:11751-11756 (2013)), as they (1) presented a diameter less than 100 nm when observed by TEM and a hydrodynamic diameter less than 200 nm when measured by DLS (FIG. 6A), (2) had a neutral or negative surface charge (FIG. 6B) and (3) were non-aggregating after incubation in artificial cerebrospinal fluid (aCSF) at 37° C. for up to 24 h (FIG. 6C).

Particles were fluorescently labeled with DiA dye, and engineered to provide comparable fluorescence intensities. For brain-penetrating NPs, the four formulations distributed widely and homogeneously when introduced into rat brains by CED, reaching distribution volumes of around 40 mm³ (FIGS. 6D and 6E)). For all particle types, distribution in the healthy brain was homogeneous through the whole caudate. These results demonstrate that all particle types were successfully engineered to exhibit similar macroscopic characteristics enabling for the controlling of any confounding variables.

### Example 5: Nanoparticles Exhibit Cellular Tropism in the Healthy Brain

Even with brain penetrating NPs, varying degrees of survival benefits in animal models were achieved (Sawyer, et al., Drug Delivery and Translational Research, 1:34-42 (2011); Zhou, et al., Proceedings of the National Academy of Sciences, 110:11751-11756 (2013); Nance, et al., ACS Nano, 8:10655-10664 (2014)), indicating that there are other variables influencing biological activity.

Previous studies of nanoparticle delivery in the brain have evaluated the macro-level interaction of NPs with tissues, by studying characteristics such as accumulation in the brain after systemic administration (Saucier-Sawyer, et al., J Drug Target, 23:736-749 (2015); Gaudin, et al., Nat Nanotechnol, 10:99 (2015)), and volume of distribution after CED (Zhou, et al., Proceedings of the National Academy of Sciences, 110:11751-11756 (2013); Mastorakos, et al., Adv Healthc Mater, 4:1023-1033 (2015); Nance, et al., ACS Nano, 8:10655-10664 (2014)), and relating these features to survival benefit. More recently, the influence of a tumor mass on the macroscopic pattern of NP distribution was investigated (Saucier-Sawyer, et al., Journal of Controlled Release Under Revision (2016)). Yet, there are few studies on how properties of NPs influence association with particular cell types once they enter the brain interstitium.

### Materials and Methods

CED of NPs in the Healthy Brain

CED of PLA NPs, PLA-HPG NPs, PLA-PEG NPs or PLA-HPG-CHO NPs in healthy Fischer 344 rats was performed as described above.

Flow Cytometry

Animals were euthanized 4 or 24 h after CED and brains were harvested. The olfactory bulb, cerebellum and contralateral hemisphere to the injection site were removed. A similar procedure was completed with isolation of the striatum, but yielded similar results; therefore the entire hemisphere was used to increase the number of counts per sample. The brain was diced into small pieces and suspended in 4 mL of PBS. Tissue was further broken down by serial pipetting and diluted to 25 mL until it passed through a 40 μm cell strainer with no resistance. After pelleting the cells, 3 mL of ACK lysing buffer (Lonza, Switzerland) was added to the suspension to lyse red blood cells. 22 mL of PBS was immediately added to neutralize the ACK buffer, and the cell suspension was then centrifuged at 950 g for 10 min and re-suspended in 4 mL of PBS. 2 mL of the cell suspension was gently placed on top of a 4 mL 20% Percoll solution (GE Healthcare Bio-Sciences, Pittsburgh, Pa.) in DMEM/F-12 and centrifuged at 3000 g for 15 min to separate out a lipid/myelin layer. This layer was removed and the rest of the solution was diluted 25× and then centrifuged for 10 min at 1000 g to collect the cells. The cells were re-suspended in 2 mL of PBS. A small aliquot was dyed with Trypan Blue and counted for cell density and viability before proceeding with staining procedures. 2 mL of 8% PFA solution was added to fix the cells for 10 min. After 2 washing steps (1000 g for 10 min), cells were resuspended in 3 mL of 0.1% triton-X solution to permeabilize for 10 min After 2 washing steps (1000 g for 10 min), non-specific binding of antibodies was prevented by blocking for 30 min in a 3 mL 10% BSA solution, followed by incubation with conjugated primary antibodies (NeuN/FOX3-Cy5 (1613R), GFAP-Cy5 (0199R), AIF-1/Iba1-Cy5 (1363R); Bioss Antibodies, Woburn, Mass.) (GFP-488, Life Technologies) at a concentration of 1 μg/250,000 cells for 30 min Finally, cells were washed three times with 1 mL of a 10% BSA solution for 10 minutes and resuspended in 320 μL of a 1% BSA solution for flow cytometry. Flow cytometry was performed using an Attune NxT (Invitrogen, Carlsbad, Calif.) with the flowing laser voltages; FSC, SSC, GFP (BL1), DiA (BL2), Cy5 (RL1): 640, 420, 400, 400, 500 respectively and 300,000 iterations were acquired. Experiments for several animals of a single particle type were done on different days to ensure reproducibility and to account for any random experimental biases.

Flow Cytometry Analysis

Data were analyzed using FlowJo v.10.0.8r1 (FlowJo, Ashland, Oreg.). After selecting for viable mononuclear cells under FSC and SSC, a population shift in the Cy-5 channel for each sample allowed for gating specific cell populations. The fluorescence histograms of the cells gated in the DiA channel were then superimposed with the histogram of the control brain cell populations, in order to gate the cell populations that shifted out of the control histogram. The mean fluorescence intensity (MFI) in the DiA channel of this final population was then recorded. MFI values were then normalized according to dye loading using the relative fluorescence of each NPs preparation obtained through a plate reader measurement. Since MFI is the measurement of the fluorescence intensity of a cell population, or (Absolute Fluorescence)/(# of cells in population), in order to deduce what the absolute fluorescence within a cell population was, the MFI had to be multiplied by the relative % of the population of cells to yield the absolute fluorescence internalized by the cells:

$$\frac{Absolute Fluorescence}{\# of cells in population} ( or M F I ) \times \frac{\# of cells in population}{Total \# of cells}$$

$$( or \% of cells in population ) .$$

Since the total # of cells for each sample was set to a fixed amount during the flow cytometry measurements, MFI x % of cells in population was proportional to the Absolute Fluorescence and could be compared between animals and samples. Finally, the sum of these fluorescence intensities within each cell population yielded the total fluorescence, or particles uptake, and was depicted through the size of the pie charts in the figures. The percent cell shift outside the control population was calculated by creating a quadrant gate in the DiA channel for the control populations.

### Immunostaining and Imaging

For immunostaining and imaging, brains were fixed in 4% PFA for 24 h and placed in a 30% sucrose solution until it equilibrated and sank. Tissue was then sliced using a Leica CM3000 Cryostat (Leica) to 10 μm slices and stored at −20° C. until staining. Just before staining, slides were rehydrated with PBS and then permeabilized with 0.1% triton-X for 1 h. The slides were then washed three times and incubated in blocking buffer (5% BSA, 5% donkey serum and 0.05% triton-X solution) for 1 h. Slides were incubated with primary antibodies (Anti Iba1 Rabbit; WAKO Pure Chemicals, Richmond, Va.), (Anti GFAP Rabbit, bs-0199R; Bioss Antibodies), (Anti NeuN Rabbit, ab1044225; Abcam, Cambridge, Mass.) diluted to 1:500 in blocking buffer for 18 h at 4° C. Slides were washed three times with blocking buffer for 10 min each and then incubated with secondary antibodies (Goat anti Rabbit Cy5, A10523; Life Technologies) diluted to 1:1000 in blocking buffer for 1 h at room temperature Finally, slides were washed two times with blocking for 10 min and once with water, then mounted for imaging using VECTASHIELD HardSet Antifade Mounting Medium with DAPI (Vector Laboratories, Burlingame, Calif.). Images were taken using a Leica TCS SP5 confocal microscope (Leica).

### Results

The brain environment is complex, with different cell types that are intimately and reciprocally linked to each other, establishing an anatomical and functional neurovascular unit (NVU) (Hawkins, et al., Pharmacol Rev, 57:173-485 (2005)), which ensures correct brain functions (Liu, et al., Life Sci, 89:141-146 (2011)). Attention was focused on three cell types known to have crucial functional roles: neurons that support information transmission; astrocytes that perform many active functions including structural and metabolic support of neurons; and microglia that represent the main cellular immune defense in the brain. Four hours after introduction of particles into the brain interstitium, the injected hemisphere was processed and analyzed by flow cytometry to measure cellular tropism of the different particle types. The three cell populations were identified with specific intracellular markers (astrocytes, microglia or neurons identified by the markers GFAP, Iba-1, and NeuN, respectively). For each formulation and each cell type, three information were extracted from the FACS data (see Methods section for detailed procedure): (1) the percentage of cells positively internalizing NPs corresponding to the population shifting from the control population, (2) the amount of NPs internalized by this population measured by the mean fluorescence intensity (MFI) (FIG. 7), and (3) taking into account the relative abundance of cells within the brain, the relative amount of NPs in each cell population, expressed as a percentage of total quantity of NPs associated with cells. Compared to the reference formulation, PLA NPs, the total NP uptake was substantially lower for PLA-PEG and PLA-HPG NPs, but significantly higher for PLA-HPG-CHO NPs (FIG. 7). More specifically, compared to PLA NPs, for which the percentage of cells that shifted out of their control population for all cell types was ˜4%, administration of PLA-PEG and PLA-HPG NPs resulted in lower percentages of cells that shifted (˜2.7% and 1.6% respectively), and administration of PLA-HPG-CHO NPs produced greater population shifts of ˜5.2%. PLA-PEG and PLA-HPG NPs distributed relatively evenly between all three cell types (FIG. 7), whereas PLA-HPG-CHO NPs presented a preferential uptake by microglia cells and decreased uptake by neurons, similar to PLA NPs.

The NP distribution among cells was analyzed using confocal microscopy. Staining for neurons (NeuN), no significant differences were observed in terms of cellular morphology. In astrocytes (GFAP), PLA-HPG-CHO NPs produced an up-regulation of GFAP protein, characteristic of reactive astrocytes (Eddleston, et al., Neuroscience, 54:15-36 (1993)). Finally, in the case of microglia (Iba-1), significant morphological differences were observed. These changes were dependent on the NP type: after introduction of PLA-PEG or PLA-HPG NPs, microglia retained a ramified shape typical of an inactivated state, whereas the presence of PLA-HPG-CHO NPs led to an amoeboid shape, typical of an activated state, similar to what was observed for PLA NPs.

To examine time-dependent cellular tropism, cellular association was quantified 24 h after particle administration. Each NP formulation was internalized more abundantly after 24 h compared to 4 h with the extent of increase being significantly influenced by the NP surface properties. PLA-PEG and PLA-HPG NPs presented a 1.4 and 2.3 fold increase, while internalization of PLA-HPG-CHO NPs was increased even more than the PLA NPs (4 and 3 fold increase respectively). These latter two NP formulations displayed different patterns regarding total population shift after 24 h. For PLA NPs, a large percentage of the cell population (22-28%) internalized a significant but relatively low amount of particles, whereas the PLA-HPG-CHO NP MFIs were increased in a smaller population of cells (6.3%-8.1%), taking up a large number of particles. These observations indicate that although global uptake was higher for PLA-HPG-CHO NPs compared to PLA NPs, it affected a smaller population of cells. Notably, as more NPs become associated with cells from 4 to 24 h, the distribution among the different cell types remained the same for all NPs formulations.

For each condition, mean fluorescence intensities of each cell population are reported. Each MFI has been normalized to the total internalization of naked PLA NPs in the healthy brain, 4 h after CED, in order to easily compare internalization levels between particle types, CED conditions (healthy brain vs tumor-bearing brain, 4 h vs 24 h), and cell type. PLA-HPG-CHO NPs displayed the highest internalization level in all conditions, while stealth particles (PLA-PEG and PLA-HPG NPs) presented similar low internalization.

Confocal imaging confirmed an increased uptake of all particle types 24 h after introduction, with the PLA-PEG and PLA-HPG NPs being internalized the least. For all NP types, the background in the particle channel was decreased and the amount of NPs in the perinuclear space of cells was increased at 24 h, with the highest intensity observed in PLA-HPG-CHO NP treated brains. Confocal images confirmed these observations regarding PLA and PLA-HPG-CHO NP internalization patterns: while PLA NPs appeared to be internalized homogeneously by a large number of cells, fewer cells took up the PLA-HPG-CHO NPs but to a greater extent. An up-regulation of GFAP proteins in brains administered with PLA NPs, and activated microglia in PLA-PEG NP treated brains was observed. On the other hand, PLA-HPG NPs did not induce activation of microglia nor did they increase the presence of reactive astrocytes, even 24 h after introduction into the brain interstitium. Overall, these results show that in the healthy brain, PLA-PEG and PLA-HPG NPs were internalized substantially less compared to PLA NPs, and conversion of diols on HPG to aldehyde groups reversed and increased uptake in all cell types.

### Example 6: Nanoparticles Exhibit Cellular Tropism in the Tumor-Bearing Brain

### Materials and Methods

Orthotopic Tumor Inoculation

Orthotopic RG2-GFP tumors were inoculated as described above. Tumors were grown for 7 days before administration of particles.

### Convection Enhanced Delivery in the Tumor Bearing Brain

CED of PLA NPs, PLA-HPG NPs, PLA-PEG NPs or PLA-HPG-CHO NPs in tumor bearing rats was conducted as described above.

### Results

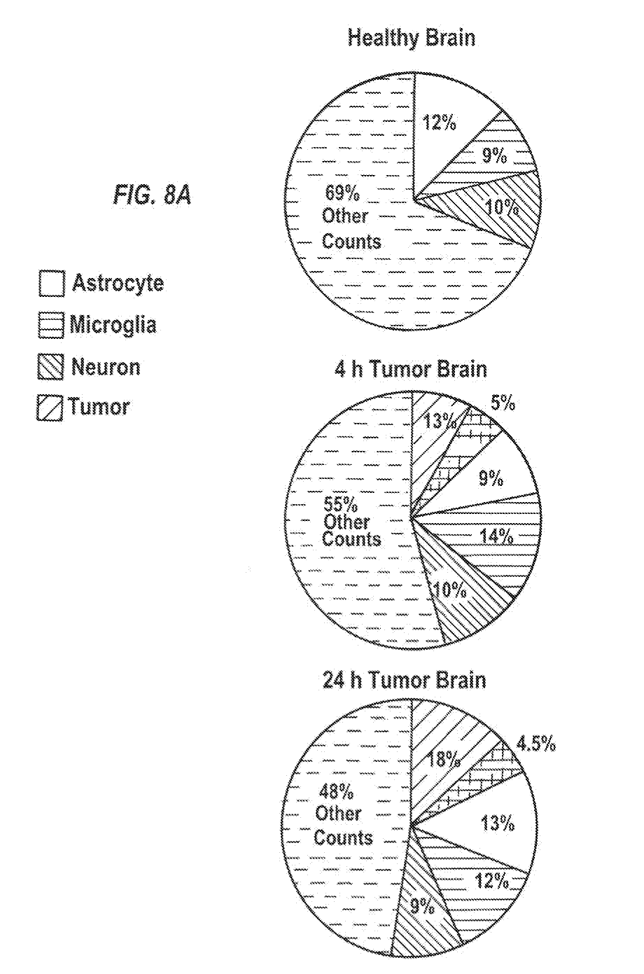

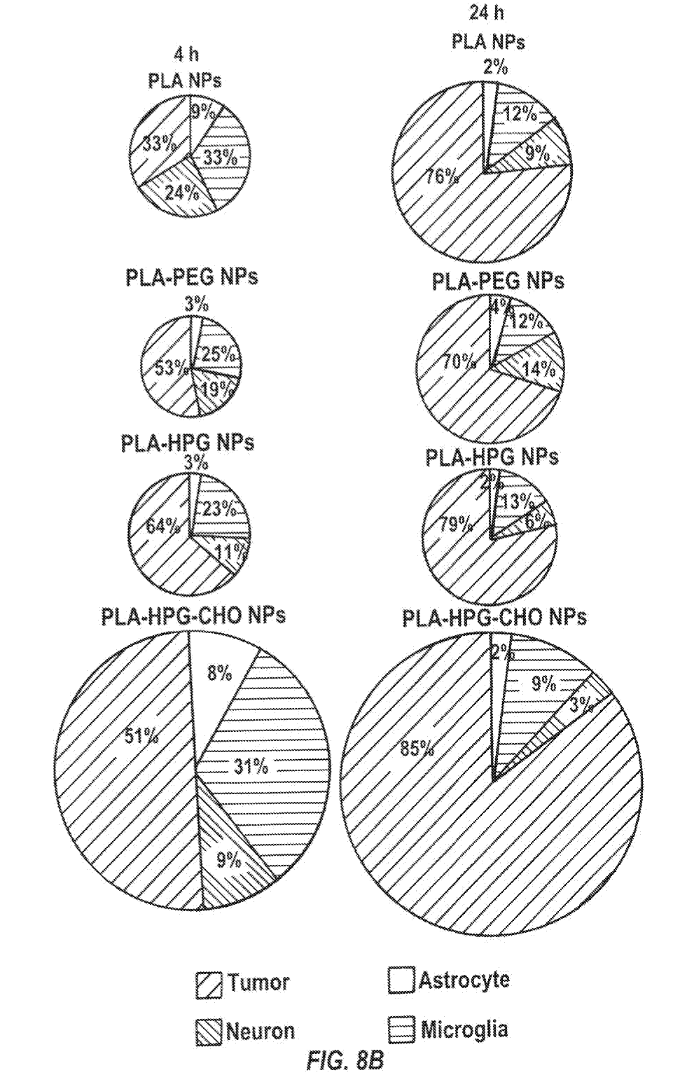

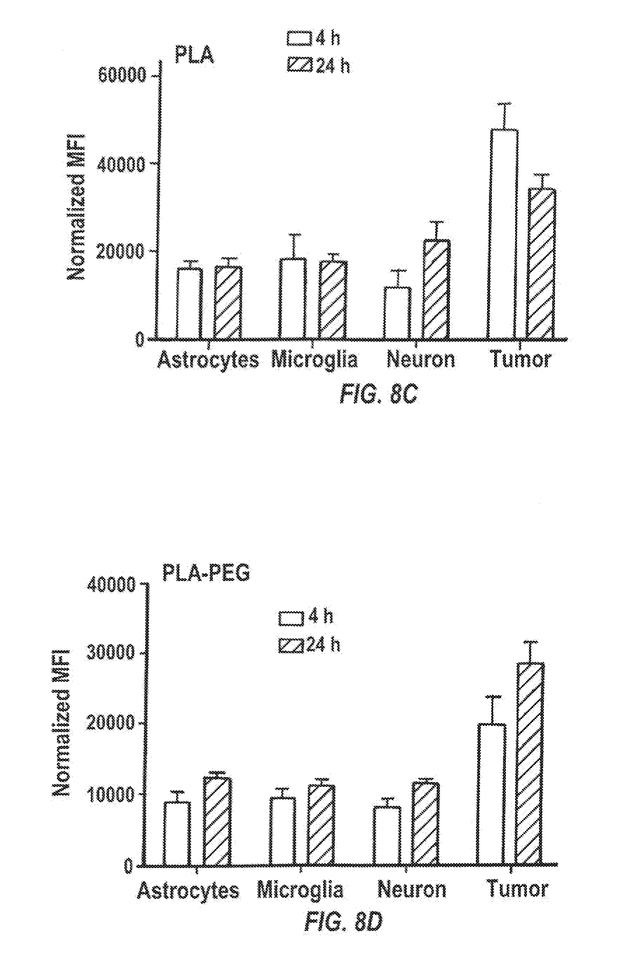

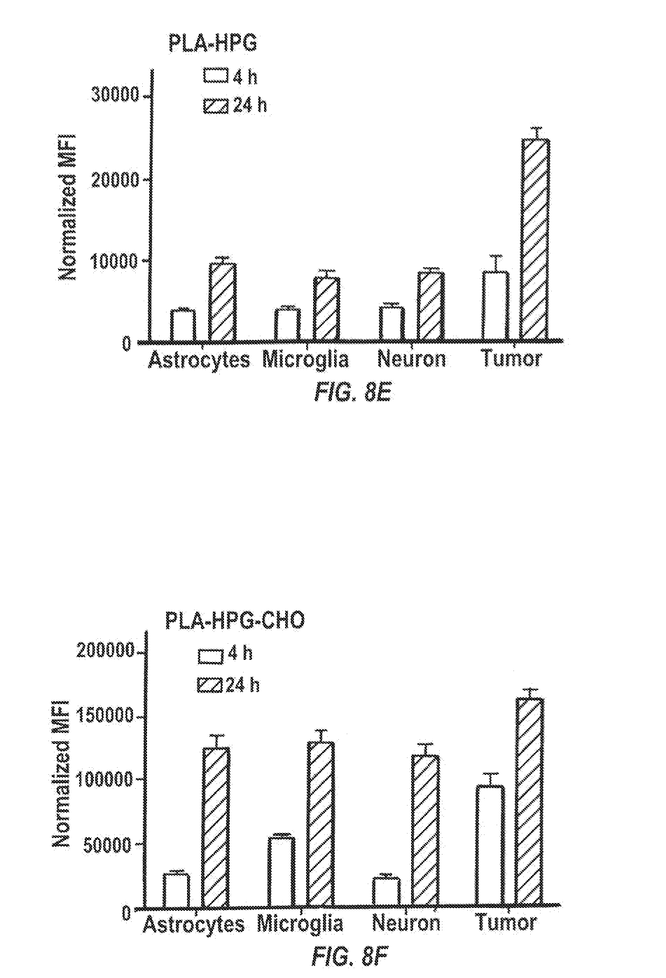

Tumor cells have been shown to strongly influence cellular interactions within the brain microenvironment, forming niches that allow for tumor protection and proliferation (Brandenburg, et al., Acta Neuropathol (2015); Calabrese, et al., Cancer Cell, 11:69-82 (2007)). They are also able to manipulate their cellular environment via secretion of proteins and display of cell surface ligands that promote tumor growth (Skog, et al., Nat Cell Biol, 10, 1470-1476 (2008)). To see if the presence of tumors influences NP fate in the brain microenvironment, similar experiments were conducted in an orthotopic brain tumor produced by injection of RG2 glioma cells. The cellular composition of the brain was first analyzed to quantify the cell populations in the tumor-free brain, compared to brains 7 or 8 days after introduction of RG2-GFP cells (FIG. 8A). At 7 or 8 days of growth, tumor cells accounted for 13 and 18% respectively of the total cell population in the hemisphere, confirming the fast development of RG2 tumors (Aas, et al., J Neurooncol, 23:175-183 (1995)). The fraction of neurons was constant among the different brains (9-10%), and consistent with literature values (Guez-Barber, et al., J Neurosci Methods, 203:10-18 (2012)). The fraction of microglia cells was slightly increased in the presence of the tumor, likely due to the recruitment of tumor-associated macrophages (TAMs) (Yi, et al., J Neuroimmunol, 232:75-82 (2011)). Finally, a sub-population of tumor cells (accounting for about 38% of the tumor population) was positive for GFAP, reflecting the astrocytic origin of RG2 tumors (Reifenberger, et al., Acta Neuropathol, 78:270-282 (1989)). Imaging of the tumor-bearing brain demonstrated the presence of activated microglia/TAMs within the tumor bulk, a strong astrogliosis at the periphery of the tumor, and the total absence of neurons inside the tumor bulk. Interestingly, within the tumor bulk, all GFAP positive cells were also GFP positive, indicating that the majority of non-tumoral cells present inside the tumor were microglia and TAMs. These observations were consistent with previous reports on rat and human GBM (Roggendorf, et al., Acta Neuropathol, 92:288-293 (1996); Placone, et al., Tumour Biol (2015)).

Infusion of NPs was performed after 7 days of tumor growth. 4 h after infusion, a significant fraction of NPs was associated with tumor cells (FIG. 8B), and this fraction varied with particle chemistry. While 32% of the tumor cells were covered with the delivery of PLA NPs, only 11% were associated with particles after the delivery of PLA-PEG or PLA-HPG NPs, and up to 76% were associated with NPs after delivery of PLA-HPG-CHO NPs. Despite the varying amounts of uptake, all three particle types presenting surface modification (PLA-PEG, PLA-HPG and PLA-HPG-CHO NPs) displayed preferential uptake by tumor cells compared to other cell types, while PLA NPs were equally internalized by tumor cells and microglia cells. Interestingly, the HPG surface modification was more efficient at decreasing microglia and neuron uptake compared to PEG, allowing for the highest specificity towards tumor cells, although the total uptake for both formulations (PLA-PEG and PLA-HPG NPs) was low.

Confocal imaging 4 h after introduction of NPs into the brain confirmed that NPs were internalized by tumor cells, microglia/TAM and GFP/GFAP positive tumor cells inside the tumor bulk, with significant uptake by activated microglia and reactive astrocytes at the tumor periphery. The extent of uptake of all NPs types was significantly increased 24 h after introduction in the interstitial space (FIGS. 8C-8F), especially for tumor cells. Once again, the fraction associated with NPs depended on their surface properties: increasing to 66% for PLA NPs, the fraction was increased only to 18% and 16% for PLA-PEG and PLA-HPG NPs respectively, and to 87% for PLA-HPG-CHO NPs. Overall, normalization of total uptake for all particle types and conditions (healthy brain vs tumor-bearing brain, 4 h vs 24 h) showed that compared to PLA NPs, PLA-HPG-CHO displayed the highest internalization in all conditions, while PLA-PEG and PLA-HPG NPs presented the lowest uptake level. The higher internalization for PLA-HPG-CHO NPs extended to all cell types, including healthy cell populations (astrocytes, microglia and neurons). These observations were further confirmed by confocal microscopy, and similarly to the healthy brain, the amount of particles in the extracellular space was reduced at 24 h compared to 4 h, whereas particles appeared concentrated in the perinuclear space.

### Example 7: In Vitro Prediction of In Vivo Cellular Tropism

For many brain diseases, the cellular target is known: tumor cells and microglia in the case of brain tumors (Wesolowska, et al., Oncogene, 27:918-930 (2008)), microglia and neurons in the case of Alzheimer's disease (Myeku, et al., Nat Med, 22:46-53 (2016)), or astrocytes in the case of amyothrophic lateral sclerosis (ALS) (Nagai, et al., Nat Neurosci, 10:615-622 (2007)). Nanoparticles are promising candidates for delivery of therapeutic agents in these settings, but there is no reliable way to determine what NP compositions are most efficient for each set of cellular targets. An in vitro screening method to identify the most relevant nanoparticle properties would be helpful.

### Materials and Methods

Cell Culture

N27 (rat neural cell) were obtained from EMD Millipore (Billercia, Mass.) and DI TNC1 (rat astrocyte) and RG2 (rat glioma) cell lines were obtained from ATCC (Manassas, Va.). BV-2 (murine microglia) cells were a kind gift from Dr. Hideyuki Takahashi from Yale Cellular Neuroscience department. N27 cells were cultured in RPMI 1640 media supplemented with 10% FBS and 1% pen/strep. DI TNC1, RG2 and BV-2 cell lines were cultured in 4.5 g/L glucose DMEM media supplemented with 10% FBS and 1% pen/strep.

Uptake Kinetic Studies

Cells were plated at a density of 10,000 cells/well in 96 well plates. 24 h after, cells were treated with fluorescent particles at a concentration of 1 mg/mL. Cells were incubated with particles for different time points (30 min, 1 h, 2 h, 4 h, 6 h, 8 h, 12 h, 24 h) washed thoroughly three times with a warm 1% BSA solution before adding trypsin and re-suspending cells in a cold 1% BSA solution on ice. Flow cytometry was performed using Attune NxT (Invitrogen) and at least 5000 iterations were acquired, then the data was analyzed using FlowJo v.10.0.8r1. Plots were fitted with the association kinetics equation on Prism 6 with the restraints assumption of dissociation rate being 0. This was done because K_{off} (off rate of particles) was assumed to be negligible in the in vitro system, allowing the direct comparison of the association rate as a predictive value of in vivo outcomes. The parameter of Hotnm (particle concentration) was not significant, because it canceled out as a normalizing factor and allowed us to derive a normalized rate of uptake of particles by cells independent of the maximum intensity. Linear regression of relative rate of uptake in vitro versus MFI values in vivo was also performed in Prism.

Graphing and Analysis

Prism 6 was used for graphing and statistical analysis. Statistical significance was tested using a two-tailed student's t-test and linear regression was analyzed using Prism 6's analysis software. Flow cytometry data analysis was done with FlowJo v.10.0.8r1 and Microsoft Office Excel 2011. Image analysis was done with MATLAB R2015a and ImageJ (FIJI plugin). Image processing was done using LAS AF (Leica) and graphical schematics were made on Adobe Photoshop CS6.

### Results

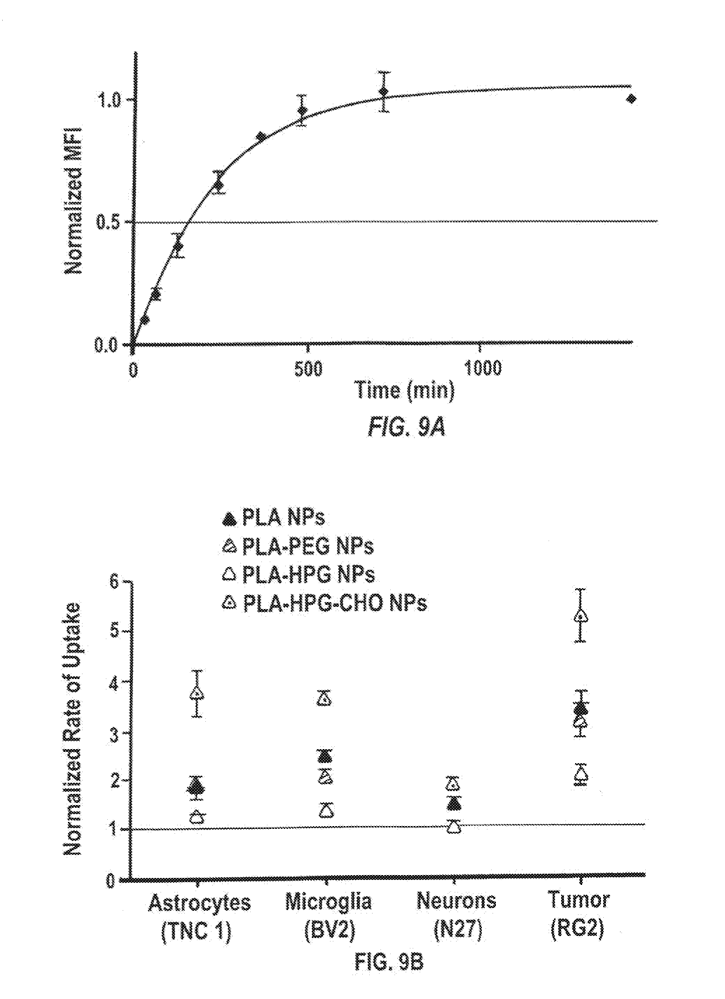

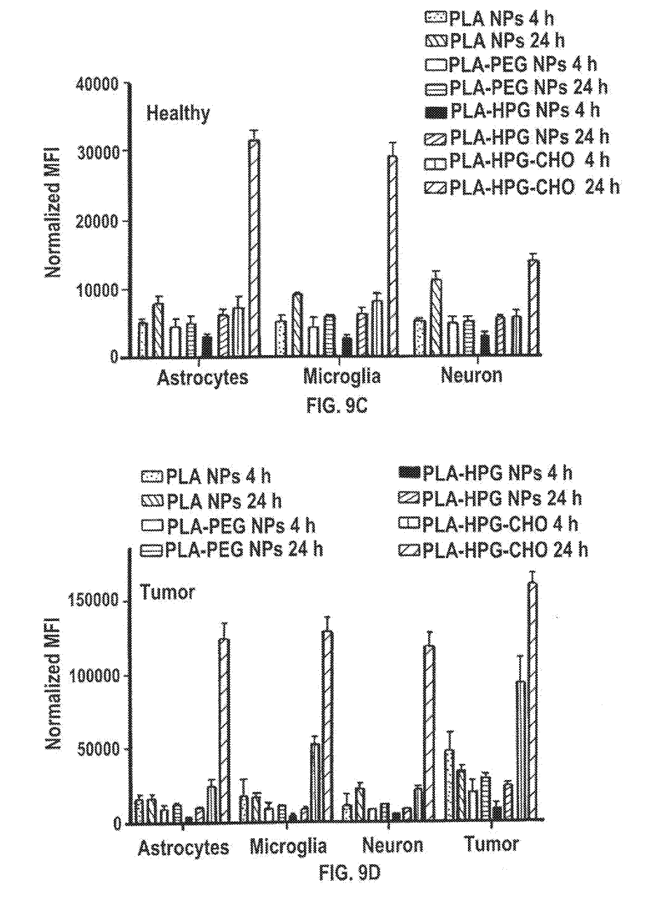

The observations of NPs association with cells in the brain were correlated with kinetic measurements of particle uptake in cultured cells (FIG. 9A, Table 2).

TABLE 2
Uptake kinetics of NPs in different cell lines.

| R² | PLA | PLA-PEG | PLA-HPG | PLA-HPG-CHO |
|---|---|---|---|---|
| values | NPs | NPs | NPs | NPs |
| TNC1 | .9024 | .9745 | .9801 | .8746 |
| BV2 | .9921 | .9758 | .9609 | .9870 |
| N27 | .9759 | .9875 | .9739 | .9816 |
| RG2 | .9111 | .9400 | .9536 | .8927 |

Since they are not decorated with any active-targeting ligands, the assumption was made that all particles were experiencing a similar uptake mechanism, likely utilizing non-specific endocytosis pathways (Lesniak, et al., J Am Chem Soc, 135:1438-1444 (2013)). Also, since the four particle formulations used in this study were engineered to have similar size, surface charge and stability in physiological conditions, along with comparable volumes of distribution when introduced into the brain, it was believed that their cellular uptake would be governed by the rate of association between the nanoparticles and the cells, likely due to differences in the protein corona acquired by the NPs as they resided in the brain interstitium (Lesniak, et al., J Am Chem Soc, 135:1438-1444 (2013); Walkey, et al., J Am Chem Soc, 134:2139-2147 (2012)). In vivo, the NPs were administered at a concentration where the system was not saturated in terms of particle concentration, so the MFI of each particle in a cell population at a given time point was used as a measurement of the rate of association of the NPs to a specific cell type (FIG. 7, 8C-8F). In vitro, NPs were delivered at a concentration that saturated the system during the 24 h experiment, so the rate of association of the NP formulation to a cell type could be derived from a simple rate equation. Therefore, it was believed that there should be a correlation between the in vivo MFI values (FIGS. 9C-9D) and the rate of uptake measured in vitro (FIG. 9B) since both were measurements of the rate of association of particles to a cell type. Indeed, when a linear regression was fit to these two values, significant slopes were observed in both healthy and tumor-bearing brains, for 4 h and 24 h time-points (P<0.001). The datafit appeared to be more predictive at 4 h compared to 24 h, both in the healthy brain (R2=0.8232 and 0.7504 at 4 h and 24 h respectively), and the tumor brain (R2=0.8397 and 0.5387 at 4 h and 24 h respectively).

Cellular tropism of NPs within tissues is critically important for their therapeutic effectiveness, as demonstrated in recent studies on the cellular distribution of particles delivered to the liver (Park, et al., Under Revision (2016)) or tumors (Ngambenjawong, et al., J Control Release, 224:103-111 (2016); Miller, et al., Sci Transl Med, 7:314 (2015)). The results indicate that the surface properties of NPs are important in determining the cellular fate of nanoparticles after entry into the brain interstitium. PEG and HPG moieties have been used to provide stealth properties in the systemic circulation (Deng, et al., Biomaterials, 35:6595-6602 (2014)), and dense PEG coating has been proposed as a means to enhance distribution in the brain interstitium (Mastorakos, et al., Adv Healthc Mater, 4:1023-1033 (2015)), indicating that stealth properties are also required for effective brain delivery. However, in the study described herein, those particles were the least efficiently internalized by all cell types, in healthy brains and in tumor-bearing brains. The lower internalization of stealth particles is likely due to their decreased retention in the brain environment resulting in reduced probability of interaction with cells and extracellular matrix making them more prone to elimination through brain capillaries (Sirianni, et al., Bioconjugate Chemistry, 25:2157-2165 (2014)) or the lymphatic system (FIGS. 9A-9D). On the other hand, when the surface diols on PLA-HPG NPs were converted to aldehydes, the NPs were internalized more abundantly by all cell types. In this example, the multifunctional HPG was modified to produce a bioadhesive state, which appears to be a valuable strategy to increase cellular uptake following brain administration.

The foregoing results also demonstrate that NP dynamics in the brain microenvironment can be modeled in cultured cells. This property may be specific to the brain environment, as introduction into the brain interstitium presents a situation in which fluid movements are slow compared to cellular uptake, which is recreated by static cell culture conditions. It is believed that the fate of a particle introduced into the brain interstitium is mainly determined by three rate-driven phenomena: (1) the cellular association rate (or set of association rates, kₒₙ) which governs the likelihood that a NP will become associated with a cell, (2) the NP clearance rate which governs the rate of loss from the interstitial space due to transport from the interstitial fluid (ISF) to the cerebrospinal fluid (CSF, k_{CSF}) or transport from the ISF to capillary blood (ksys), and (3) the cell division rate (−kₘᵢₜ), which determines the rate at which the number of cells able to internalize NPs increase, effectively changing the concentration of particles that are available per cell. These three phenomena and their relative contributions to the fate of a NP population are dependent on the characteristics of the brain microenvironment: for example, in the healthy brain, the mitotic activity of the resident cells is very low, such that the third rate parameter may be negligible, whereas in the tumor brain, highly mitotic and metabolic tumor cells make this rate important.

The in vivo experimental data supported this model. Between 4 h and 24 h after introduction of NPs into the brain interstitium, the increase of total uptake varied between particle types but the relative uptake by the different cell types remained unchanged for all formulations. This indicates that in an environment where cells have low mitotic or metabolic activity, surface properties of the NPs drive cellular association, which remains comparable over time. In tumor-bearing brains, mitotically active tumor cells, and their ability to activate microglia and to recruit TAMs, strongly influenced NP uptake depending on their surface properties. For example, NP association with tumor cells increased from 4 to 24 h by 34%, 7%, 5% and 11% for PLA, PLA-PEG, PLA-HPG and PLA-HPG-CHO NPs, respectively. For these intracranial tumors, the tumor cell content increased by 5% from day 7 to 8, indicating that the increase in PLA-PEG and PLA-HPG NPs uptake was mainly due to cellular multiplication, while PLA and PLA-HPG-CHO NPs actively interacted with the highly metabolic tumor cells.

This study demonstrates that NP surface properties influence cellular tropism after their entry into the brain, and that engineering these surface properties provides an opportunity to control cellular distribution. In particular, stealth formulations were shown to escape cellular uptake both in the healthy brain and the tumor-bearing brain. On the other hand, introduction of bioadhesive surface modifications can dramatically enhance the association of NPs with particular cell populations, such as tumor cells. Further, in vitro rates of association can indicate in vivo cellular affinity, showing that the dynamic and complex brain environment can be modeled, at least to some extent, by a simple static in vitro system. Altogether, these results highlight the potential to optimize NP-based therapeutic strategies by tuning NP surface properties for enhanced delivery to certain cell types of interest, which can be associated with improved therapeutic efficacy in target cells and minimized treatment toxicity to off-target cells.

1. A formulation for delivering therapeutic, prophylactic or diagnostic agents to the central nervous system comprising nanoparticles loaded with a therapeutic, prophylactic or diagnostic agent and consisting essentially of an average diameter of less than 100 nm, wherein the nanoparticles comprise a hydrophobic core comprising hydrophobic polymer or molecule and a hyperbranched polymeric shell.

2. The formulation of claim 1, wherein the nanoparticle core comprises a hyperbranched polyglycerol.

3. The formulation of claim 2, wherein the surface hydroxyl groups of the hyperbranched polyglycerol were converted to aldehydes.

4. The formulation of claim 1, wherein the hydrophobic polymeric core is a polyester.

5. The formulation of claim 1, wherein the nanoparticles comprise one or more targeting moieties.

6. The formulation of claim 5, wherein at least one of the targeting moieties targets an adenosine receptor.

7. The formulation of claim 6, wherein the targeting moiety that targets the adenosine receptors is an adenosine agonist.

8. The formulation of claim 7, wherein the adenosine agonist is selected from the group consisting of Exemplary agonists include, but are not limited to, (2R,3R,4S,5R)-2-(6-amino-9H-purin-9-yl)-5-(hydroxymethyl)oxolane-3,4-diol (adenosine), 4-[2-[[6-Amino-9-(N-ethyl-β-D-ribofuranuronamidosyl)-9H-purin-2-yl]amino]ethyl]benzenepropanoic acid hydrochloride (CGS 21680), N6-cyclo-hexyladenosine (CHA), 2-Chloro-N-cyclopentyladenosine (CCPA), 2-Chloro-N-cyclopentyl-2′-methyladenosine (2′-MeCCPA), N-Cyclopentyladenosine (CPA), 3-[4-[2-[[6-amino-9-[(2R,3R,4S,5S)-5-(ethylcarbamoyl)-3,4-dihydroxy-oxolan-2-yl]purin-2-yl]amino]ethyl]phenyl]propanoic acid (CGS21680), 2-(1-Hexynyl)-N-methyladenosine (HEMADO), 2-chloro-N6-(3-iodobenzyl)adenosine-5′-N-methylcarboxamide (Cl-IB-MECA), 1-[2-Chloro-6-[[(3-iodophenyl)methyl]amino]-9H-purin-9-yl]-1-deoxy-N-methyl-β-D-ribofuranuronamide (2-Cl-IB-MECA), 1-Deoxy-1-[6-[[(3-iodophenyl)methyl]amino]-9H-purin-9-yl]-N-methyl-β-D-ribofuranuronamide (IB-MECA), [2-[6-Amino-3,5-dicyano-4-[4-(cyclopropylmethoxy)-phenyl]pyridin-2-ylsulfanyl]acetamide] (BAY606583), 5′-N-Ethylcarboxamidoadenosine (NECA), and N-Cyclohexyl-2′-O-methyladenosine (SDZ WAG 994).

9. The formulation of claim 8, wherein the adenosine agonist is adenosine.

10. The formulation of claim 5, wherein at least one of the targeting moieties is pHLIP.

11. The formulation of claim 1, wherein the nanoparticles have at least one of the following characteristics selected from the group consisting of (i) an average diameter less than about 100 nm when observed by transmission electron microscopy (TEM), (ii) a hydrodynamic diameter less than about 200 nm when measured by dynamic light scattering (DLS), (iii) a neutral or negative surface charge, and (iv) non-aggregating after incubation in artificial cerebrospinal fluid (aCSF) at 37° C. for up to 24 hours.

12. The formulation of claim 1 further comprising trehalose, glucose, sucrose, lactose, mannitol or a combination thereof in an effective amount to reduce aggregation of the nanoparticles.

13. The formulation of claim 1, wherein the agent is a nucleic acid or a small molecule drug.

14. The formulation of claim 13, wherein the agent is an inhibitory nucleic acid.

15. The formulation of claim 13, wherein the agent is a small molecule chemotherapeutic agent.

16. The formulation of claim 15 wherein the agent is effective for the treatment or alleviation of one or more symptoms of a neurodegenerative disease, disorder or injury to the CNS.

17. A method of delivering a therapeutic, prophylactic or diagnostic agent to the central nervous system comprising administering into the blood stream or tissue adjacent to the region of the central nervous system to be treated the formulation of claim 1.

18. The method of claim 17, wherein the formulation is administered to the brain by convection enhanced delivery.

19. The method of claim 17, wherein the subject has brain tumors or a neurodegenerative disease.

20. The method of claim 17, wherein the subject has a brain cancer.

21. A method of treating brain cancer comprising convection enhanced delivery of the formulation of claim 1 to the brain of a subject with brain tumors, wherein the formulation is administered in an effective amount to reduce tumor size or burden.


SEQUENCE LISTING (2 sequences)

SEQ ID NO: 1 - protein, 37 aa, Artificial Sequence
  synthetic polypeptide pH-low insertion peptide
AAEQNPIYWARYADWLFTTPLLLLDLALLVDADEGTG

SEQ ID NO: 2 - protein, 36 aa, Artificial Sequence
  synthetic polypeptide pH-low insertion peptide
GGEQNPIYWARYADWLFTTPLLLLDLALLVDADEGT